(19) United States
(12) Patent Application Publication
(10) Pub. No.: US 2009/0292220 A1
(43) Pub. Date: Nov. 26, 2009

(54) RESPIRATION MONITORING APPARATUS, RESPIRATION MONITORING SYSTEM, MEDICAL PROCESSING SYSTEM, RESPIRATION MONITORING METHOD AND RESPIRATION MONITORING PROGRAM

(75) Inventor: Yoshio Miyake, Tokyo (JP)

Correspondence Address: TUROCY & WATSON, LLP, 127 Public Square, 57th Floor, Key Tower, CLEVELAND, OH, 44114, US

(73) Assignees: KABUSHIKI KAISHA TOSHIBA, Tokyo (JP); TOSHIBA SOLUTIONS CORPORATION, Tokyo (JP)

(21) Appl. No.: 12/092,378

(22) PCT Filed: Nov. 2, 2006
(86) PCT No.: PCT/JP2006/321986

§ 371 Date: May 1, 2008

(30) Foreign Application Priority Data
Nov. 4, 2005 (JP) 2005-320343

(51) Int. Cl. A61B 5/08

(52) U.S. Cl. 600/534

(57) ABSTRACT

A respiration monitoring apparatus includes an image acquiring section that acquires an image of an imaging target region including a physical part of a subject that reciprocates in response to the respiration of the subject as picked up with inclination of a predetermined angle relative to the reciprocating direction at each predetermined timing, a displacement computing section that computationally determines the displacement of the position of the imaging target region between the first clock time that is an arbitrarily selected timing and the second clock time that is the timing of a predetermined number of counts as counted from the first clock time on the basis of the difference between the luminance of each of the pixels on the image acquired at the first clock time and the luminance of the corresponding pixel on the image acquired at the second clock time, and a position determining section that determines the position obtained by adding the displacement of the position of the imaging target region between the first clock time and the second clock time as computationally determined by the displacement computing section to the position of the imaging target region at the first clock time as the position of the imaging target region at the second clock time.

### TECHNICAL FIELD

The present invention relates to a respiration monitoring process for grasping the respiring condition of a subject.

### BACKGROUND ART

The technique referred to as retrospective gating scan is being utilized to pick up an image of a subject typically by means of CT scan. Retrospective gating scan is a technique of scanning an internal organ that moves in response to breathing (e.g., lung, liver or spleen) in a phase of the repetitive breathing cycle of expiration and inspiration to make it possible to pick up images of the internal organ that are in phase.

Then, as a result, it is possible to acquire images of a part of the body with reduced motion artifacts if the part is apt to be influenced by respiratory motion artifacts and provides difficulties of acquiring sharp images.

For an operation of retrospective gating scan as described above, it is necessary to grasp the positional displacement of a position on the body surface produced by the reciprocating motion of the thorax or the abdomen of the subject due to respiration.

With conventional retrospective gating scan, it is a general practice to put a device for detecting the tension produced by respiration to a part of the body (e.g., a position near the thorax or the abdomen) in order to grasp the displacement of a position on the body surface produced at the time of expiration or inspiration of the subject.

### DISCLOSURE OF THE INVENTION

### Problem to be Solved by the Invention

However, the above-described prior art is accompanied by a problem including that the subject is forced to feel uncomfortable by the device that is directly put to a part of his or her body in order to detect the displacement of a position on the body surface produced by respiration and that the device inevitably enters into the angle of view of the CT scan apparatus that scans the subject.

The present invention dissolves the above-identified problem of the prior art by providing a technique that allows to grasp the degree of displacement of a position on the body surface produced at the time of expiration or respiration of the subject without contacting the subject.

### Means for Solving the Problem

In an aspect of the present invention, the above problem is solved by providing a respiration monitoring apparatus including: an image acquiring section that acquires an image of an imaging target region including a physical part of a subject that reciprocates in response to the respiration of the subject as picked up with inclination of a predetermined angle relative to the reciprocating direction at each predetermined timing; a displacement computing section that computationally determines the displacement of the position of the imaging target region between the first clock time that is an arbitrarily selected timing and the second clock time that is the timing of a predetermined number of counts as counted from the first clock time on the basis of the difference between the luminance of each of the pixels on the image acquired at the first clock time and the luminance of the corresponding pixel on the image acquired at the second clock time; and a position determining section that determines the position obtained by adding the displacement of the position of the imaging target region between the first clock time and the second clock time as computationally determined by the displacement computing section to the position of the imaging target region at the first clock time as the position of the imaging target region at the second clock time.

Preferably, in the respiration monitoring apparatus having the above-described configuration, when the coordinate value of a pixel in a direction substantially parallel to the height direction of the subject is y, the coordinate value of the pixel in a direction substantially perpendicular to the height direction of the subject is x, the first clock time is t₁, the second clock time is t₂ and the luminance value of the pixel of the coordinates (x, y) at clock time t is I(x, y, t) in the imaging target region on the image acquired by the image acquiring section, the displacement Q of the position of the imaging target region between the first clock time and the second clock time responding to the respiration of the subject is computationally determined by formula Q=S S|(I(x, y, t₂)−I(x, y, t₁))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

Preferably, in the respiration monitoring apparatus having the above-described configuration, the image acquiring section is adapted to determine the moving direction of the pixels of image from the temporal displacement of the pixels on the images as acquired at a plurality of predetermined consecutive timings. The respiration monitoring apparatus further includes an expiration/inspiration determining section that determines the movement of the pixels on the images as that of inspiration when they move in the first direction as directional component on a plane defined by the height direction of the subject and a direction substantially parallel to the transversal direction relative to the subject but determines the movement of the pixels as that of expiration when they move in the second direction substantially opposite to the first direction, and the position determining section is adapted to add the displacement of the position of the imaging target region as displacement in expiration or in inspiration according to the outcome of determination by the expiration/inspiration determining section.

Preferably, in the respiration monitoring apparatus having the above-described configuration, when the coordinate value of a pixel in a direction substantially parallel to the height direction of the subject is y, the coordinate value of the pixel in a direction substantially perpendicular to the height direction of the subject is x, the clock time is t and the luminance value of the pixel of the coordinates (x, y) at clock time t is I(x, y, t) in the imaging target region on the image acquired by the image acquiring section, the speed dy/dt of all the pixels in the imaging target region in the direction substantially parallel to the height direction of the subject is given by dy/dt=−(−S S((∂I(x, y, t)∂x)*(∂I(x, y, t)/∂y))*S S((∂I(x, y, t)/∂t)*(∂I(x, y, t)/∂y))+S S(∂I(x, y, t)/∂x)²*S S((∂I(x, y, t)∂y)*(∂I(x, y, t)/∂t)))/S S(∂I(x, y, t)/∂y)²*S S(∂I(x, y, t)/∂x)²−(S S((∂I(x, y, t)/∂x)*(∂I(x, y, t)/∂y))²), (where the first S of the S S is the sum of the luminance values of all the pixels either in the y direction or in the x direction in the imaging target region, the second S of the S S is the sum of the luminance values of all the pixels either in the x direction or in the y direction, whichever appropriate, in the imaging target region), and the expiration/inspiration determining section is adapted to determine that the direction of the speed of dy/dt indicates inspiration when it is directed in the first direction on the image and that it indicates expiration when it is directed in the second direction on the image.

In another aspect of the present invention, there is provided a respiration monitoring apparatus including: an image acquiring section that acquires an image of an imaging target region including a physical part of a subject that reciprocates in response to the respiration of the subject as picked up with inclination of a predetermined angle relative to the reciprocating direction at each predetermined timing; a displacement computing section that computationally determines the displacement of the position of the imaging target region between an arbitrary clock time that is an arbitrarily selected timing and a reference clock time that is the timing for the imaging target region to get to a predetermined limit position in the respiration prior to the arbitrary clock time on the basis of the difference between the luminance of each of the pixels on the image acquired at the arbitrary clock time and the luminance of the corresponding pixel on the image acquired at the reference clock time; and a position determining section that determines the displacement of the position of the imaging target region between the reference clock time and the arbitrary clock time as computationally determined by the displacement computing section as the position of the imaging target region at the arbitrary clock time.

Preferably, in the respiration monitoring apparatus having the above-described configuration, when the coordinate value of a pixel in a direction substantially parallel to the height direction of the subject is y, the coordinate value of the pixel in a direction substantially perpendicular to the height direction of the subject is x, the reference clock time is t₀, the arbitrary clock time is tₙ and the luminance value of the pixel of the coordinates (x, y) at clock time t is I(x, y, t) in the imaging target region on the image acquired by the image acquiring section, the displacement Q_{b} of the position of the imaging target region between the reference clock time and the arbitrary clock time responding to the respiration of the subject is computationally determined by formula Q_{b}=S S|(I(X, y, tₙ)−I(x, y, t₀))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

Preferably, the respiration monitoring apparatus having the above-described configuration further includes an imaging region defining section that defines the region having a predetermined number of pixels that maximizes the temporal change of luminance of the pixels on the image obtained by shooting the subject as the imaging target region.

In still another aspect of the present invention, there is provided a respiration monitoring apparatus including: an image acquiring section that acquires an image of an imaging target region including a physical part of a subject that reciprocates in response to the respiration of the subject as picked up with inclination of a predetermined angle relative to the reciprocating direction at each predetermined timing; a displacement computing section that extracts the second region having pixels showing a luminance distribution substantially same as the first region having a plurality of arbitrarily selected pixels in the imaging target region on the image acquired by the image acquiring section at the first clock time that is an arbitrarily selected timing from the imaging target region on the image acquired at the second clock time that is the timing of a predetermined number of counts as counted from the first clock time and computationally determines the distance of movement from the position of the first region to the position of the second region in the imaging target region as the displacement of the position of the imaging target region from the first clock time to the second clock time; and a position determining section that determines the position obtained by adding the displacement of the position of the imaging target region between the first clock time and the second clock time as computationally determined by the displacement computing section to the position of the imaging target region at the first clock time as the position of the imaging target region at the second clock time.

In still another aspect of the present invention, there is provided a respiration monitoring apparatus including: an image acquiring section that acquires an image of an imaging target region including a physical part of a subject that reciprocates in response to the respiration of the subject as picked up with inclination of a predetermined angle relative to the reciprocating direction at each predetermined timing; a displacement computing section that extracts from the imaging target region on the image acquired by the image acquiring section at an arbitrary clock time that is an arbitrarily selected timing the second region having pixels showing a luminance distribution substantially same as the first region having a plurality of arbitrarily selected pixels in the imaging target region on the image acquired by the image acquiring section at a reference clock time that is the timing for the imaging target region to get to a predetermined limit position in the respiration prior to the arbitrary clock time and computationally determines the distance of movement from the position of the first region to the position of the second region in the imaging target region as the displacement of the position of the imaging target region from the reference clock time to the arbitrary clock time; and a position determining section that determines the displacement of the position of the imaging target region between the reference clock time and the arbitrary clock time as computationally determined by the displacement computing section as the position of the imaging target region at the arbitrary clock time.

Preferably, the respiration monitoring apparatus having the above-described configuration further includes an imaging region defining section that defines the imaging target region as a region centered at a pixel region that maximizes the temporal change of luminance of the pixels on the image obtained by shooting the subject.

In still another aspect of the present invention, there is provided a respiration monitoring system including: a respiration monitoring apparatus that has a configuration as defined above; and an image pickup section that picks up an image of an imaging target region from a position located obliquely above relative to the imaging target region at the side of the feet of the subject lying on the back.

In still another aspect of the present invention, there is provided a medical processing system including a respiration monitoring apparatus that has a configuration as defined above; and a medical process executing section that causes a predetermined medical process to be executed when the position of the imaging target region as determined by the position determining section is located at a predetermined position.

In the medical processing system having a configuration as described above, the predetermined medical process is an imaging process to be executed by means of MRI or CT scan.

In still another aspect of the present invention, there is provided a respiration monitoring method including: an image acquiring step that acquires an image of an imaging target region including a physical part of a subject that reciprocates in response to the respiration of the subject as picked up with inclination of a predetermined angle relative to the reciprocating direction at each predetermined timing; a displacement computing step that computationally determines the displacement of the position of the imaging target region between the first clock time that is an arbitrarily selected timing and the second clock time that is the timing of a predetermined number of counts as counted from the first clock time on the basis of the difference between the luminance of each of the pixels on the image acquired at the first clock time and the luminance of the corresponding pixel on the image acquired at the second clock time; and a position determining step that determines the position obtained by adding the displacement of the position of the imaging target region between the first clock time and the second clock time as computationally determined in the displacement computing step to the position of the imaging target region at the first clock time as the position of the imaging target region at the second clock time.

Preferably, in the respiration monitoring method of the above-described arrangement, when the coordinate value of a pixel in a direction substantially parallel to the height direction of the subject is y, the coordinate value of the pixel in a direction substantially perpendicular to the height direction of the subject is x, the first clock time is t₁, the second clock time is t₂ and the luminance value of the pixel of the coordinates at clock time t is I(x, y, t) in the imaging target region on the image acquired in the image acquiring step, the displacement Q of the position of the imaging target region between the first clock time and the second clock time responding to the respiration of the subject is computationally determined by formula Q=S S|(I(x, y, t₂)−I(x, y, t₁))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

Preferably, in the respiration monitoring method of the above-described arrangement, the image acquiring step is adapted to determine the moving direction of the pixels of image from the temporal displacement of the pixel in the images as acquired at a plurality of predetermined consecutive timings. The respiration monitoring method further includes an expiration/inspiration determining step that determines the movement of the pixels on the images as that of inspiration when they move in the first direction as directional component on a plane defined by the height direction of the subject and a direction substantially parallel to the transversal direction relative to the subject but determines the movement of the pixels as that of expiration when they move in the second direction substantially opposite to the first direction, and the position determining step is adapted to add the displacement of the position of the imaging target region as displacement in expiration or inspiration according to the outcome of determination in the expiration/inspiration determining step.

Preferably, in the respiration monitoring method of the above-described arrangement, when the coordinate value of a pixel in a direction substantially parallel to the height direction of the subject is y, the coordinate value of the pixel in a direction substantially perpendicular to the height direction of the subject is x, the clock time is t and the luminance value of the pixel of the coordinates (x, y) at clock time t is I(x, y, t) in the imaging target region on the image acquired in the image acquiring step, the speed dy/dt of all the pixels in the imaging target region in the direction substantially parallel to the height direction of the subject is given by dy/d=−(−S S((∂I(x, y, t)/∂x)*(∂I(x, y, t)/∂y))*S S((∂I(x, y, t)/∂t)*(∂I(x, y, t)/∂y))+S S((∂I(x, y, t)/∂x)²*S S((∂I(x, y, t)/∂y)*(∂I(x, y, t)/∂t)))/S S(∂I(x, y, t)/∂y)²*S S(∂I(x, y, t)/∂x)²−(S S((∂I(x, y, t)/∂x)*(∂I(x, y, t)/∂y))²), (where the first S of the S S is the sum of the luminance values of all the pixels either in the y direction or in the x direction in the imaging target region, the second S of the S S is the sum of the luminance values of all the pixels either in the x direction or in the y direction, whichever appropriate, in the imaging target region) and the expiration/inspiration determining step is adapted to determine that the direction of the speed of dy/dt indicates inspiration when it is directed in the first direction on the image and that it indicates expiration when it is directed in the second direction on the image.

In another aspect of the present invention, there is provided a respiration monitoring method including: an image acquiring step that acquires an image of an imaging target region including a physical part of a subject that reciprocates in response to the respiration of the subject as picked up with inclination of a predetermined angle relative to the reciprocating direction at each predetermined timing; a displacement computing step that computationally determines the displacement of the position of the imaging target region between an arbitrary clock time that is an arbitrarily selected timing and a reference clock time that is the timing for the imaging target region to get to a predetermined limit position in the respiration prior to the arbitrary clock time on the basis of the difference between the luminance of each of the pixels on the image acquired at the arbitrary clock time and the luminance of the corresponding pixel on the image acquired at the reference clock time; and a position determining step that determines the displacement of the position of the imaging target region between the reference clock time and the arbitrary clock time as computationally determined in the displacement computing step as the position of the imaging target region at the arbitrary clock time.

Preferably, in the respiration monitoring method of the above-described arrangement, when the coordinate value of a pixel in a direction substantially parallel to the height direction of the subject is y, the coordinate value of the pixel in a direction substantially perpendicular to the height direction of the subject is x, the reference clock time is t₀, the arbitrary clock time is tₙ and the luminance value of the pixel of the coordinates (x, y) at clock time t is I(x, y, t) in the imaging target region on the image acquired in the image acquiring step, the displacement Q_{b} of the position of the imaging target region between the reference clock time and the arbitrary clock time responding to the respiration of the subject is computationally determined by formula Q_{b}=S S|(I(X, y, tₙ)−I(X, y, t₀))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

Preferably, the respiration monitoring method of the above-described arrangement further includes an imaging region defining step that defines the region having a predetermined number of pixels that maximizes the temporal change of luminance of the pixels on the image obtained by shooting the subject as the imaging target region.

In still another aspect of the present invention, there is provided a respiration monitoring method including: an image acquiring step that acquires an image of an imaging target region including a physical part of a subject that reciprocates in response to the respiration of the subject as picked up with inclination of a predetermined angle relative to the reciprocating direction at each predetermined timing; a displacement computing step that extracts the second region having pixels showing a luminance distribution substantially same as the first region having a plurality of arbitrarily selected pixels in the imaging target region on the image acquired in the image acquiring step at the first clock time that is an arbitrarily selected timing from the imaging target region on the image acquired at the second clock time that is the timing of a predetermined number of counts as counted from the first clock time and computationally determines the distance of movement from the position of the first region to the position of the second region in the imaging target region as the displacement of the position of the imaging target region from the first clock time to the second clock time; and a position determining step that determines the position obtained by adding the displacement of the position of the imaging target region between the first clock time and the second clock time as computationally determined in the displacement computing step to the position of the imaging target region at the first clock time as the position of the imaging target region at the second clock time.

In still another aspect of the present invention, there is provided a respiration monitoring method including: an image acquiring step that acquires an image of an imaging target region including a physical part of a subject that reciprocates in response to the respiration of the subject as picked up with inclination of a predetermined angle relative to the reciprocating direction at each predetermined timing; a displacement computing step that extracts from the imaging target region on the image acquired in the image acquiring step at an arbitrary clock time that is an arbitrarily selected timing the second region having pixels showing a luminance distribution substantially same as the first region having a plurality of arbitrarily selected pixels in the imaging target region on the image acquired at a reference clock time that is the timing for the imaging target region to get to a predetermined limit position in the respiration prior to the arbitrary clock time and computationally determines the distance of movement from the position of the first region to the position of the second region in the imaging target region as the displacement of the position of the imaging target region from the reference clock time to the arbitrary clock time; and a position determining step that determines the displacement of the position of the imaging target region between the reference clock time and the arbitrary clock time as computationally determined in the displacement computing step as the position of the imaging target region at the arbitrary clock time.

Preferably, the respiration monitoring method of the above-described arrangement further includes an imaging region defining step that defines the imaging target region as a region centered at a pixel region that maximizes the temporal change of luminance of the pixels on the image obtained by shooting the subject.

In still another aspect of the present invention, there is provided a respiration monitoring program for causing a computer to execute: an image acquiring step that acquires an image of an imaging target region including a physical part of a subject that reciprocates in response to the respiration of the subject as picked up with inclination of a predetermined angle relative to the reciprocating direction at each predetermined timing; a displacement computing step that computationally determines the displacement of the position of the imaging target region between the first clock time that is an arbitrarily selected timing and the second clock time that is the timing of a predetermined number of counts as counted from the first clock time on the basis of the difference between the luminance of each of the pixels on the image acquired at the first clock time and the luminance of the corresponding pixel on the image acquired at the second clock time; and a position determining step that determines the position obtained by adding the displacement of the position of the imaging target region between the first clock time and the second clock time as computationally determined in the displacement computing step to the position of the imaging target region at the first clock time as the position of the imaging target region at the second clock time.

Preferably, in the respiration monitoring program of the above-described arrangement, when the coordinate value of a pixel in a direction substantially parallel to the height direction of the subject is y, the coordinate value of the pixel in a direction substantially perpendicular to the height direction of the subject is x, the first clock time is t₁, the second clock time is t₂ and the luminance value of the pixel of the coordinates (x, y) at clock time t is I(x, y, t) in the imaging target region on the image acquired in the image acquiring step, the displacement Q of the position of the imaging target region between the first clock time and the second clock time responding to the respiration of the subject is computationally determined by formula Q=S S|(I(x, y, t₂)−I(x, y, t₁))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

Preferably, in the respiration monitoring program of the above-described arrangement, the image acquiring step is adapted to determine the moving direction of the pixels of image from the temporal displacement of the pixel in the images as acquired at a plurality of predetermined consecutive timings. The respiration monitoring method further includes an expiration/inspiration determining step that determines the movement of the pixels on the images as that of inspiration when they move in the first direction as directional component on a plane defined by the height direction of the subject and a direction substantially parallel to the transversal direction relative to the subject but determines the movement of the pixels as that of expiration when they move in the second direction substantially opposite to the first direction, and the position determining step is adapted to add the displacement of the position of the imaging target region as displacement in expiration or inspiration according to the outcome of determination in the expiration/inspiration determining step.

Preferably, in the respiration monitoring program of the above-described arrangement, when the coordinate value of a pixel in a direction substantially parallel to the height direction of the subject is y, the coordinate value of the pixel in a direction substantially perpendicular to the height direction of the subject is x, the clock time is t and the luminance value of the pixel of the coordinates (x, y) at clock time t is I(x, y, t) in the imaging target region on the image acquired in the image acquiring step, the speed dy/dt of all the pixels in the imaging target region in the direction substantially parallel to the height direction of the subject is given by dy/dt=−(−S S((∂I(x, y, t)∂x)*(∂I(x, y, t)/∂y))*S S((∂I(x, y, t)/∂t)*(∂I(x, y, t)/∂y))+S S(∂I(x, y, t)/∂x)²*S S((∂I(x, y, t)/∂y)*(∂I(x, y, t)/∂t)))/(S S(∂I(x, y, t)/∂y)²*S S(∂I(x, y, t)/∂x)²−(S S(∂I(x, y, t)/∂x)*(∂I(x, y, t)/∂y))²), (where the first S of the S S is the sum of the luminance values of all the pixels either in the y direction or in the x direction in the imaging target region, the second S of the S S is the sum of the luminance values of all the pixels either in the x direction or in the y direction, whichever appropriate, in the imaging target region) and the expiration/inspiration determining step is adapted to determine that the direction of the speed of dy/dt indicates inspiration when it is directed in the first direction on the image and that it indicates expiration when it is directed in the second direction on the image.

In another aspect of the present invention, there is provided a respiration monitoring program for causing a computer to execute: an image acquiring step that acquires an image of an imaging target region including a physical part of a subject that reciprocates in response to the respiration of the subject as picked up with inclination of a predetermined angle relative to the reciprocating direction at each predetermined timing; a displacement computing step that computationally determines the displacement of the position of the imaging target region between an arbitrary clock time that is an arbitrarily selected timing and a reference clock time that is the timing for the imaging target region to get to a predetermined limit position in the respiration prior to the arbitrary clock time on the basis of the difference between the luminance of each of the pixels on the image acquired at the arbitrary clock time and the luminance of the corresponding pixel on the image acquired at the reference clock time; and a position determining step that determines the displacement of the position of the imaging target region between the reference clock time and the arbitrary clock time as computationally determined in the displacement computing step as the position of the imaging target region at the arbitrary clock time.

Preferably, in the respiration monitoring program of the above-described arrangement, when the coordinate value of a pixel in a direction substantially parallel to the height direction of the subject is y, the coordinate value of the pixel in a direction substantially perpendicular to the height direction of the subject is x, the reference clock time is t₀, the arbitrary clock time is tₙ and the luminance value of the pixel of the coordinates at clock time t is I(x, y, t) in the imaging target region on the image acquired in the image acquiring step, the displacement Q_{b} of the position of the imaging target region between the reference clock time and the arbitrary clock time responding to the respiration of the subject is computationally determined by formula Q_{b}=S S|((X, y, tₙ)−I(X, y, t₀))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

Preferably, the respiration monitoring program of the above-described arrangement further includes an imaging region defining step that defines the region having a predetermined number of pixels that maximizes the temporal change of luminance of the pixels on the image obtained by shooting the subject as the imaging target region.

In still another aspect of the present invention, there is provided a respiration monitoring program for causing a computer to execute: an image acquiring step that acquires an image of an imaging target region including a physical part of a subject that reciprocates in response to the respiration of the subject as picked up with inclination of a predetermined angle relative to the reciprocating direction at each predetermined timing; a displacement computing step that extracts the second region having pixels showing a luminance distribution substantially same as the first region having a plurality of arbitrarily selected pixels in the imaging target region on the image acquired in the image acquiring step at the first clock time that is an arbitrarily selected timing from the imaging target region on the image acquired at the second clock time that is the timing of a predetermined number of counts as counted from the first clock time and computationally determines the distance of movement from the position of the first region to the position of the second region in the imaging target region as the displacement of the position of the imaging target region from the first clock time to the second clock time; and a position determining step that determines the position obtained by adding the displacement of the position of the imaging target region between the first clock time and the second clock time as computationally determined in the displacement computing step to the position of the imaging target region at the first clock time as the position of the imaging target region at the second clock time.

In a further aspect of the present invention, there is provided a respiration monitoring program for causing a computer to execute: an image acquiring step that acquires an image of an imaging target region including a physical part of a subject that reciprocates in response to the respiration of the subject as picked up with inclination of a predetermined angle relative to the reciprocating direction at each predetermined timing; a displacement computing step that extracts from the imaging target region on the image acquired in the image acquiring step at an arbitrary clock time that is an arbitrarily selected timing the second region having pixels showing a luminance distribution substantially same as the first region having a plurality of arbitrarily selected pixels in the imaging target region on the image acquired at a reference clock time that is the timing for the imaging target region to get to a predetermined limit position in the respiration prior to the arbitrary clock time and computationally determines the distance of movement from the position of the first region to the position of the second region in the imaging target region as the displacement of the position of the imaging target region from the reference clock time to the arbitrary clock time; and a position determining step that determines the displacement of the position of the imaging target region between the reference clock time and the arbitrary clock time as computationally determined in the displacement computing step as the position of the imaging target region at the arbitrary clock time.

Preferably, the respiration monitoring program of the above-described arrangement further includes an imaging region defining step that defines the imaging target region as a region centered at a pixel region that maximizes the temporal change of luminance of the pixels on the image obtained by shooting the subject.

### BRIEF DESCRIPTION OF THE DRAWINGS

FIG. 1 is a schematic functional block diagram illustrating a respiration monitoring apparatus, a respiration monitoring system and a medical processing system according to the first embodiment of the present invention;

FIG. 2 is a schematic illustration of the relationship between the location for setting up the image pickup section 2 and the movement of the pixels in the ROI according to the vertical movement of the thorax or the abdomen of the subject due to respiration;

FIG. 3 is another schematic illustration of the relationship between the location for setting up the image pickup section 2 and the movement of the pixels in the ROI according to the vertical movement of the thorax or the abdomen of the subject due to respiration;

FIG. 4 is a flowchart of the process of respiration monitoring apparatus (reproduction monitoring method) according to the first embodiment;

FIG. 5 is a detailed flowchart of the process of the expiration/inspiration determining section 104;

FIG. 6 is a schematic exemplar image showing the position of an imaging target region as displayed on the display section 106;

FIG. 7 is a flowchart of the process of the respiration monitoring apparatus (respiration monitoring method) according to the second embodiment of the present invention;

FIG. 8 is a flowchart of the process of the respiration monitoring apparatus (respiration monitoring method) according to the third embodiment of the present invention;

FIG. 9 is a flowchart of the process of determining the moving directions of pixels on an image to be executed by the expiration/inspiration determining section 104 of the third embodiment;

FIG. 10 is another flowchart of the process of determining the moving directions of pixels on an image to be executed by the expiration/inspiration determining section 104 of the third embodiment;

FIG. 11 is a schematic illustration of the movement of a predetermined block on an image and a method of matching the block;

FIG. 12 is another schematic illustration of the movement of a predetermined block on an image and a method of matching the block;

FIG. 13 is a schematic exemplar graph showing the position determined by the position determining section 105 as displayed on the display section 106; and

FIG. 14 is a flowchart of the process of the respiration monitoring apparatus (respiration monitoring method) according to the fourth embodiment of the present invention.

### BEST MODE FOR CARRYING OUT THE INVENTION

### First Embodiment

Now, the first embodiment of the present invention will be described by referring to the related drawings.

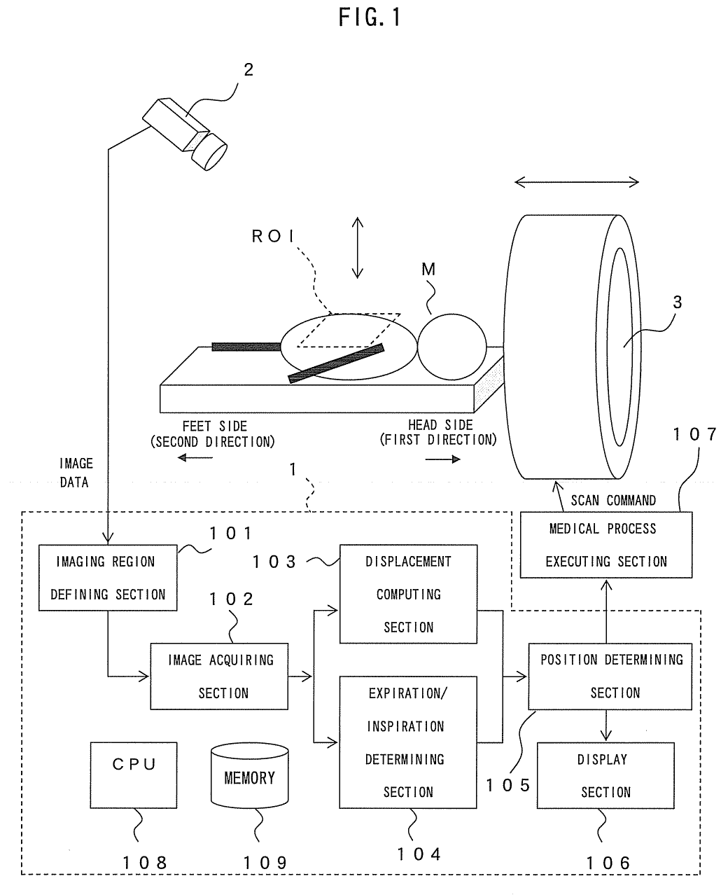

FIG. 1 is a functional block diagram illustrating a respiration monitoring apparatus, a respiration monitoring system and a medical processing system according to first embodiment of the present invention.

The respiration monitoring apparatus 1 of this embodiment includes an imaging region defining section 101, an image acquiring section 102, a displacement computing section 103, an expiration/inspiration determining section 104, a position determining section 105, a display section 106, a CPU 108 and a MEMORY 109. The medical processing system of this embodiment includes a medical process executing section 107 and a CT scan apparatus 3 in addition to a respiration monitoring apparatus 1 as described above. The respiration monitoring system of this embodiment includes a respiration monitoring apparatus as described above and an image pickup section 2.

The image pickup section 2 typically includes a CCD camera and takes a role of picking up an image of an imaging target region ROI that includes at least either the thorax or the abdomen of subject M (namely, an imaging target region including a physical part that moves back and forth in response to the respiration of the subject) with inclination of a predetermined angle relative to the imaging target region. More specifically, as shown in FIG. 1, the image pickup section 2 shoots the imaging target region ROI obliquely from above from the side of the feet of the subject M who is lying on the back.

The imaging region defining section 101 takes a role of defining a region on the image obtained by shooting the subject M that has a predetermined number of pixels and in which the change with time of the luminance of the pixels is maximized as imaging target region ROI.

The image acquiring section 102 takes a role of acquiring an image of the imaging target region including a physical part that reciprocates in response to the respiration of the subject with inclination of a predetermined angle at each predetermined timing.

The displacement computing section 103 takes a role of computationally determining the displacement of the position of the imaging target region between the first clock time that is an arbitrary selected timing and the second clock time that is the timing of a predetermined number of counts as counted from the first clock time (the displacement of the body surface due to expiration or inspiration) on the basis of the difference between the luminance of each of the pixels on the image acquired by the image acquiring section 102 at the first clock time and the luminance of the corresponding pixel on the image acquired also by the image acquiring section 102 at the second clock time. The second clock time is selected according to the processing capacity of the CPU and/or the intervals of frames of the image that can be acquired by the image acquiring section 102 of the respiration monitoring apparatus 1. For instance, when it is desirable to highly accurately monitor the respiration of the subject, the selected second clock time may be the timing by which a single count is counted. On the other hand, when the accuracy of monitoring the respiration does not give rise to any problem if a relatively large number is selected for the counts, the selected second clock time may be the timing by which two or three counts are counted.

The expiration/inspiration determining section 104 takes a role of determining the moving direction of the pixels of image from the temporal displacement of the pixels on the images on the basis of the plurality of images acquired by the image acquiring section 102 at a plurality of predetermined consecutive timings such that it determines the movement of the pixels on the images as that of inspiration when they move in the first direction (on the image) as directional component on a plane defined by the height direction of the subject M and a direction substantially parallel to the transversal direction relative to the height direction of the subject M but determines the movement of the pixels as that of expiration when they move in the second direction (on the image) substantially opposite to the first direction. The expression of a plane defined by the height direction of the subject M and a direction substantially parallel to the transversal direction relative to the subject refers to a substantially horizontal plane when the subject M is lying on the back as shown in FIG. 1.

Since the image pickup section 2 is adapted to shoot the imaging target region ROI obliquely from above from the side of the feet of the subject M who is lying on the back, the expiration/inspiration determining section 104 determines that the movement of the pixels on the image toward the head side of the subject M (in the first direction) as the movement due to inspiration and that of the pixels on the image toward the feet side of the subject M (in the second direction) as the movement due to expiration.

The position determining section 105 takes a role of determining the position of the imaging target region obtained by adding the displacement of the imaging target region between the position of the imaging target region at the first clock time and that of the imaging target region at the second clock time as computationally determined by the displacement computing section 103 to the position of the imaging target region at the first clock time as the position of the imaging target region at the second clock time. Additionally, the position determining section 105 adds the displacement of the position of the imaging target region as displacement in expiration or in inspiration according to the outcome of determination by the expiration/inspiration determining section 104.

The display can 106 includes a liquid crystal display or a CRT display and takes a role of displaying various pieces of information relating to the processes of the respiration monitoring apparatus 1 such as the outcome of determination by the position determining section 105 on a display screen.

The medical process executing section 107 takes a role of driving the CT scan apparatus 3 to execute an image pickup process as a predetermined medical process when the position of the imaging target region is located at a predetermined position as determined by the position determining section 105.

The CPU 108 takes a role of executing various processes of the respiration monitoring apparatus and also that of realizing various functions by executing programs stored in the MEMORY 109. The MEMORY 109 typically includes a ROM and a RAM and takes a role of storing various pieces of information and programs to be utilized in the respiration monitoring apparatus.

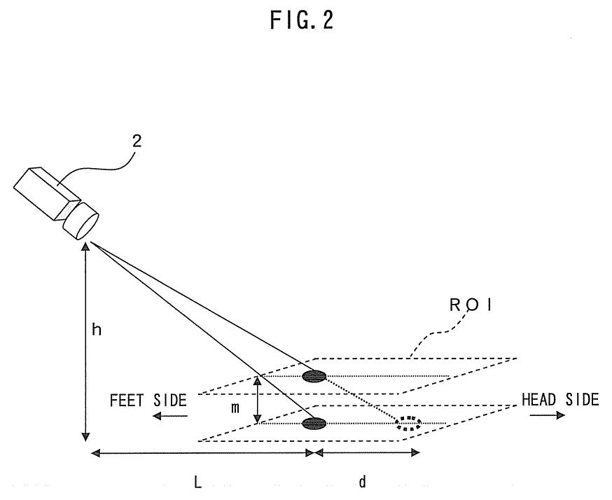

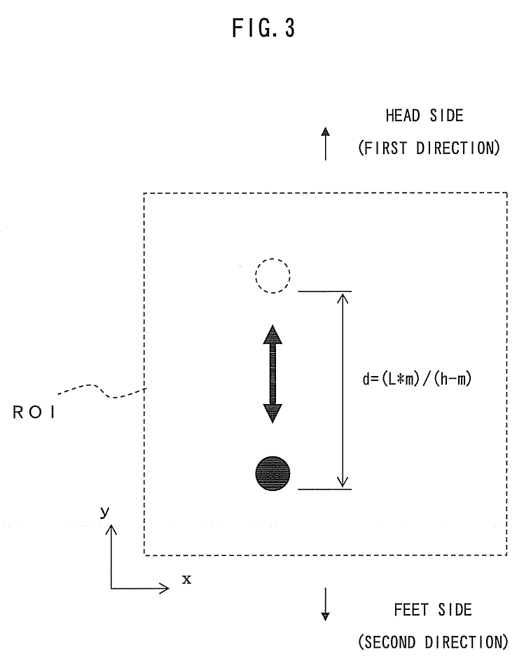

Now, the principle of computationally determining the displacement of the body surface produced as a result of respiration by means of the respiration monitoring apparatus of this embodiment will be described below. FIGS. 2 and 3 are schematic illustrations of the relationship between the location for setting up the image pickup section 2 and the movement of the pixels in the ROI according to the vertical movement of the thorax or the abdomen of the subject due to respiration.

As shown in FIGS. 2 and 3, when the thorax or the abdomen of a subject M that is defined as imaging target region ROI is shot by the image pickup section 2, an arbitrarily selected point on the thorax or the abdomen may appear to be moving up and down as the subject M breathes on the image picked up by the image pickup section 2. In other words, the vertical movement of the thorax or the abdomen of the subject M produced as a result of respiration can be determined according to the displacement of the pixels on the image picked up by the image pickup section 2.

More specifically, when the distance from the image pickup section 2 to the thorax of the abdomen of the subject M that is defined as imaging target region ROI in the vertical direction is h and the distance from the image pickup section 2 to an arbitrarily selected point on the thorax or the abdomen of the subject M in the horizontal direction is L, the displacement d of each pixel on the image picked up by the image pickup section 2 is expressed by

d(=(L×m)/(h−m)) (1).

In this embodiment, the displacement of the thorax of the abdomen due to a vertical movement thereof produced by respiration is grasped on the basis of the movement of the pixels in the ROI.

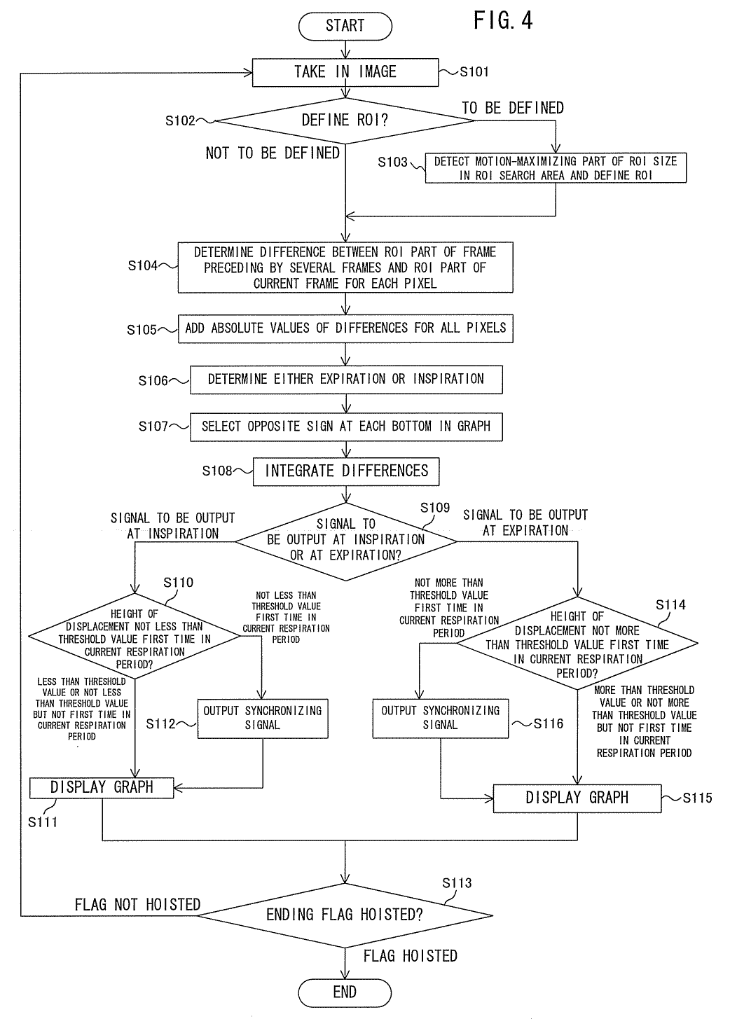

FIG. 4 is a flowchart of the process (the reproduction monitoring method) of the first embodiment of respiration monitoring apparatus.

Firstly, the image pickup section 2 shoots the physical part that reciprocates in response to the respiration of the subject M and its neighboring area (S101).

When an imaging target region ROI is to be defined (S102, to be defined), the region of a predetermined number of pixels that maximizes the change with time of the luminance of the pixels on the image picked up by the image pickup section 2 by shooting the subject M (the predetermined number of pixels corresponding to the size of the ROI) is defined as imaging target region (imaging region defining step) (S103). The image acquiring section 102 acquires an image picked up so as to show an inclination of a predetermined angle relative to the reciprocating direction of the defined imaging target region at each predetermined timing (image acquiring step).

Subsequently, the difference between the luminance of each of the pixels on the image acquired by the image acquiring section 102 at the first clock time that is an arbitrarily selected timing (the ROI of the frame preceding the current frame by several frames) and the luminance of the corresponding pixel on the image acquired by the image acquiring section 102 at the second clock time that is that is the timing of a predetermined number of counts as counted from the first clock time (the ROI of the current frame) is computationally determined (S104) and the absolute values of the differences of all the pixels obtained as a result of the above process are added (S105). Then, the displacement of the position of the imaging target region from the first clock time to the second clock time is computationally determined (displacement computing step).

Now, the process of the displacement computing section 103 will be described below in detail. When the coordinate value of a pixel in a direction substantially parallel to the height direction of the subject M is y, the coordinate value of the pixel in a direction substantially perpendicular to the height direction of the subject M is x, the first clock time is t₁, the second clock time is t₂ and the luminance value of the pixel of the coordinates (x, y) at clock time t is I(x, y, t) in the imaging target region ROI on the image acquired by the image acquiring section 102, the displacement Q of the position of the imaging target region (more specifically, the surface of the body in the imaging target region ROI) between the first clock time and the second clock time responding to the respiration of the subject is computationally determined by formula:

Q=S S|(I(x,y,t₂)−I(x,y,t₁))| (2),

(where the first S of the S S is the sum of the luminance values of all the pixels either in the y direction or in the x direction in the imaging target region ROI and the second S of the S S is the sum of the luminance values of all the pixels either in the x direction or in the y direction, whichever appropriate, in the imaging target region ROI). The displacement computing section 103 of this embodiment computationally determines the displacement of the position of the imaging target region from the first clock time to the second clock time according to the formula (2) above.

In order to grasp the displacement of a position on the body surface due to respiration on the basis of the displacement computationally determined by the displacement computing section 103 in the above-described manner, it is necessary to determine if the displacement is due to expiration or inspiration. Thus, the expiration/inspiration determining section 104 determines the moving direction of the pixels of image from the temporal displacement of the pixels on the images as acquired at a plurality of predetermined consecutive timings by the image acquiring section 102 and then determines that the movement of the pixels on the images as that of inspiration when they move in the first direction as directional component on a plane defined by the height direction of the subject M and a direction substantially parallel to the transversal direction relative to the subject but determines that the movement of the pixels as that of expiration when they move in the second direction substantially opposite to the first direction (expiration/inspiration determining step) (S106).

Now, the process of the expiration/inspiration determining section 104 will be described in greater detail. If a movement of the body is due to expiration or inspiration can be determined on the basis of the movement of the pixels on the picked up image. The method of determining the moving direction of the pixels on the image picked up by the image pickup section 2 that the expiration/inspiration determining section 104 uses will be described below.

When the coordinate value of a pixel in a direction substantially parallel to the height direction of the subject M is y, the coordinate value of the pixel in a direction substantially perpendicular to the height direction of the subject M is x, the clock time is t and the luminance value of the pixel of the coordinates (x, y) at clock time t is I(x, y, t) in the imaging target region on the image acquired by the image acquiring section 102, the following equation holds true because the pixels on the image of a physical part to be examined move to other positions after a short period of time (dt) as a result of respiration.

I(x,y,t)=I(x+dx,y+dy,t+dt) (3)

The right side of the above equation is subjected to Tailor expansion and the terms of the higher order of dx, dy, dt are disregarded as they are very small. Then, the following equation is obtained by dividing the two sides by dt.

(dx/dt)*∂I(x,y,t)/∂x+(dy/dt)*∂I(x,y,t)/∂y+∂I(x,y,t)/∂t=∂t=0 (4)

Since the changes of speed of neighboring pixels at a time can be regarded to be substantially equal to each other, there exists an equation that minimizes the error of the expression of the left side for all the neighboring pixels. In other words, when E=S S((dx/dt)*∂I(x, y, t)/∂x+(dy/dt)*∂I(x, y, t)/∂y+∂I(x, y, t)/∂t)², u=dx/dt and v=dy/dt are assumed, the two equations of ∂E/∂u=0 and ∂E/∂v=0 hold true. From these equations, the speed dy/dt of all the pixels can be determined by the formula shown below.

dy/dt=−(−S S((∂I(x,y,t)/∂x)*(∂I(x,y,t)/∂y))*S S((∂I(x,y,t)/∂t)*(∂I(x,y,t)/∂y))+S S(∂I(x,y,t)/∂x)²*S S((∂I(x,y,t)/∂y)*(∂I(x,y,t)/∂t)))/S S(∂I(x,y,t)/∂y)²*S S(∂I(x,y,t)/∂x)²−(S S((∂I(x,y,t)/∂x)*(∂I(x,y,t)/∂y))²) (5)

In the above formula, the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region.

The vertical displacement of the image of the physical part to be examined (ROI) can be obtained by computationally determining the solution of the above formula within the scope of the image of the imaging target region ROI. Then, the displacement is determined to be that of inspiration when the physical part to be examined is displaced upward (toward the head) as a result of the computation, whereas it is determined to be that of expiration when the physical part to be examined is displaced downward (toward the feet) as a result of the computation (in other words, if the head side is “+” and the feet side is “−” in the y direction, the displacement is determined to be that of inspiration when dy/dt takes a positive value, whereas it is determined to be that of expiration when dy/dt takes a negative value).

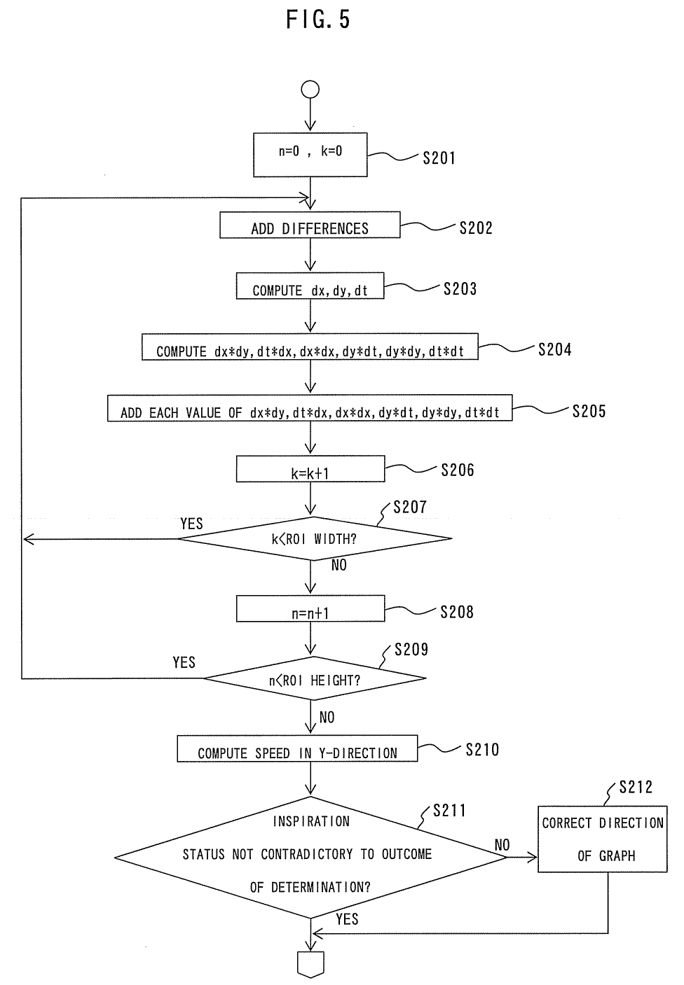

FIG. 5 is a detailed flowchart of the process of the expiration/inspiration determining section 104.

Referring to FIG. 5, firstly the indexes n and k of the pixels on the image are initialized (S201).

Then, a difference-adding process of adding the differences of the luminance values of a pixel (identified by the index n) of image among the images acquired at a plurality of predetermined consecutive timings (a process of adding the absolute values of the differences obtained by computing the differences of the luminance values among the frames for each pixel) is executed by the image acquiring section 102 (S202).

Subsequently, the difference dx of the luminance values of pixels in the X-direction, the difference dy of the luminance values of pixels in the Y-direction and the difference dt of the luminance values of each of the same pixels at the same positions among the frames acquired at a plurality of predetermined consecutive timings are computed (S203).

Thereafter, the values of dx×dy, dt×dx, dx×dx, dy×dt, dy×dy, and dt×dt are computed on the basis of the values determined for dx, dy and dt in the above step (S203) (S204).

Then, the obtained results of the above step (S204) are added for each of the formulas of the above-described step (S204) (S205).

Thereafter, the index k of each pixel for the X-direction is incremented by 1 in the X-direction (S206) and if the index k for the X-direction exceeds the corresponding limit of the imaging target region ROI in the X-direction or not is checked (S207).

If the index k of pixel for the X-direction exceeds the corresponding limit of the imaging target region ROI in the X-direction (S207, No), the index n of pixel for the Y-direction is incremented by 1 (S208).

Then, if the index n of pixel for the Y-direction exceeds the corresponding limit of the imaging target region ROI in the Y-direction or not is checked (S209).

In this way, the process of adding the differences of the luminance values of pixels is executed so long as the indexes of each pixel are found within the limits of the imaging target region ROI.

Then, the temporal displacement (in terms of speed and direction) in the Y-direction of each of all the pixels in the imaging target region ROI is computationally determined according to the above-described formula (1) (S210).

Then, if the pixels in the imaging target region ROI are moving as a whole in the direction toward the head or in the direction toward the feet is determined according to the temporal displacements in the Y-direction of all the pixels computed in the above-described step (S210) (S211). At this time, it is also determined if the current status of respiration is contradictory to the obtained outcome of determination or not.

For example, if the current status of respiration is that of inspiration and the pixels in the imaging target region ROI are moving as a whole in the direction toward the head, the process proceeds to the operation of processing the pixels of the next frame (the frame of image acquired at the next timing relative to the frame that is the current object of judgment) (S201).

If, on the other hand, the current status of respiration is that of inspiration and the pixels in the imaging target region ROI are moving as a whole in the direction toward the feet, the current status of respiration is contradictory to the obtained outcome so that the outcome of determination is corrected from inspiration to “expiration” (S212).

Then, the computed displacement is differentiated between expiration and inspiration by means of the sign prefixed to the quantity of displacement on the basis of the outcome of the determining process executed by the expiration/inspiration determining section 104 in order to discriminate the displacement computed by the displacement computing section 103 between expiration and inspiration (S107).

Subsequently, the position determining section 105 determines the position obtained by adding the displacement of the position of the imaging target region (as the displacement produced by an expiratory motion or an inspiratory motion) between the first clock time and the second clock time as computationally determined in the displacement computing step to the position of the imaging target region at the first clock time (at the timing preceding the second clock time by several frames) according to the outcome of determination of the expiration/inspiration determining step as the position of the imaging target region at the second clock time (the image acquiring timing of the current frame) (position determining step) (S108). Note that the position of the imaging target region as determined in the position determining step indicates the ratio of the body surface at the second clock time relative to the limit of the variation of the body surface of the thorax or the abdomen produced by a cycle of respiration of the subject as expressed by %.

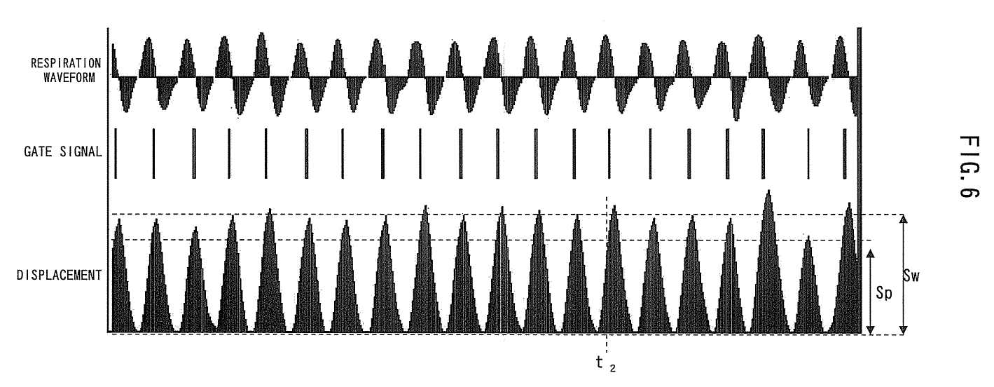

Then, the medical process executing section 107 checks if the timing of issuing a scan command to the CT scan apparatus 3 is at the time of an expiratory motion or that of an inspiratory motion (S109). When a scan command is to be issued at the time of an inspiratory motion (S109, to output a signal at the time of an inspiratory motion) and if the position as determined by the position determining section 105 exceeds a threshold value for the first time in the period of a cycle of respiration from inspiration to expiration (S110, exceeds the threshold value for the first time in the current respiration period), the medical process executing section 107 issues a scan command to the CT scan apparatus 3 (medical process executing step) (S112). After issuing the scan command, the medical process executing section 107 has the display section 106 display a graph showing the position as determined by the position determining section 105 (S111). If, on the other hand, the position as determined by the position determining section 105 does not exceed the threshold value or exceeds the threshold value not for the first time, the medical process executing section 107 has the display section 106 display a graph showing the position as determined by the position determining section 105(S111). FIG. 6 is a schematic exemplar image showing the position of an imaging target region as displayed on the display section 106. As shown in FIG. 6, the positional change of the imaging target region due to the respiration of the subject appears as “displacement”.

When, on the other hand, a scan command is to be issued at the time of an expiratory motion (S109, to output a signal at the time of an expiratory motion) and if the position as determined by the position determining section 105 falls below a threshold value for the first time in the period of a cycle of respiration from inspiration to expiration (S114, falls below the threshold value for the first time in the current respiration period), the medical process executing section 107 issues a scan command to the CT scan apparatus 3 (S116). After issuing the scan command, the medical process executing section 107 has the display section 106 display a graph showing the position as determined by the position determining section 105 (S115). If, on the other hand, the position as determined by the position determining section 105 does not fall below the threshold value or falls below the threshold value not for the first time, the medical process executing section 107 has the display section 106 display a graph showing the position as determined by the position determining section 105 (S115).

Note that, for example, the threshold value to be compared with the position determined by the position determining section 105 is defined as the position Sp of 70% of the amplitude average value Sw relative to the position of the amplitude average value Sw where the subject completely breathes out in the waveform obtained from the information on the position as determined by the position determining section 105 for a predetermined period of time (the displacement waveform of the body surface of the thorax or the abdomen and the surroundings thereof as shown in FIG. 6. Thus, the “position” as determined by the position determining section 105 refers to the position as expressed by % relative to the amplitude of the displacement waveform of the body surface of the thorax or the abdomen and the surroundings thereof.

A graph showing the position as determined by the position determining section 105 is displayed (S111, S115) and when the process is ended (S113, a flag is hoisted), the process is ended. If, on the other hand, the process is not ended (S113, no flag is hoisted), the process returns to the operation of acquiring an image once again (S101).

As described above, this embodiment can grasp the displacement of the position of the body surface of the thorax or the abdomen produced by the respiration of the subject in a non-contact manner and hence can execute a medical process, specifying the timing at which the body surface gets to an arbitrarily selected position in the respiration (e.g., the thorax expands to what % position in a cycle of respiration).

Additionally, when this embodiment is so adapted as to determine the position of the imaging target region on the basis of the change in the luminance of the pixels in the ROI, it can determine the position of the imaging target region as long as the difference of luminance value can be detected. Then, for example, it is possible to determine the position of the imaging target region if the subject is wearing a plain garment whose luminance changes only delicately.

### Second Embodiment

Now, the second embodiment of the present invention will be described below.

Since this embodiment is a modification to the above-described first embodiment, the component same as those of the above-described first embodiment are denoted by the same reference symbols and will not be described here any further. This embodiment differs from the above-described first embodiment in terms of the method of computing the displacement that the displacement computing section employs.

The displacement computing section 103 of this embodiment is so adapted as to computationally determine the displacement of the position of the imaging target region between an arbitrary clock time that is an arbitrarily selected timing and a reference clock time that is the timing for the imaging target region to get to a predetermined limit position in the respiration prior to the arbitrary clock time on the basis of the difference between the luminance of each of the pixels on the image acquired by the image acquiring section 102 at the arbitrary clock time and the luminance of the corresponding pixel on the image acquired by the image acquiring section 102 at the reference clock time.

The expression that the imaging target region gets to a predetermined limit position in the respiration refers to the timing when the subject completely breathes in an inspiratory motion or the timing when the subject completely breathes out in an expiratory motion. Then, as a result, the displacement of the position of the imaging target region after the timing when the subject completely breathes in can be determined as a displacement produced by an expiratory motion, whereas the displacement of the position of the imaging target region after the timing when the subject completely breathes out can be determined as a displacement produced by an inspiratory motion. In other words, expiration and inspiration can be discriminated without requiring any special process by defining a timing that operates as reference as in the case of this embodiment.

The position determining section 105 of this embodiment is adapted to determine the displacement of the position of the imaging target region between the reference clock time and an arbitrary clock time as determined by the displacement computing section 103 as the position of the imaging target region at the arbitrary clock time. In other words, while the position determining section of the above-described first embodiment determines the position of the imaging target region by incrementally adding the displacement as computed by the displacement computing section 103, the position determining section of this embodiment determines the position of the imaging target region as absolute position on the basis of a reference.

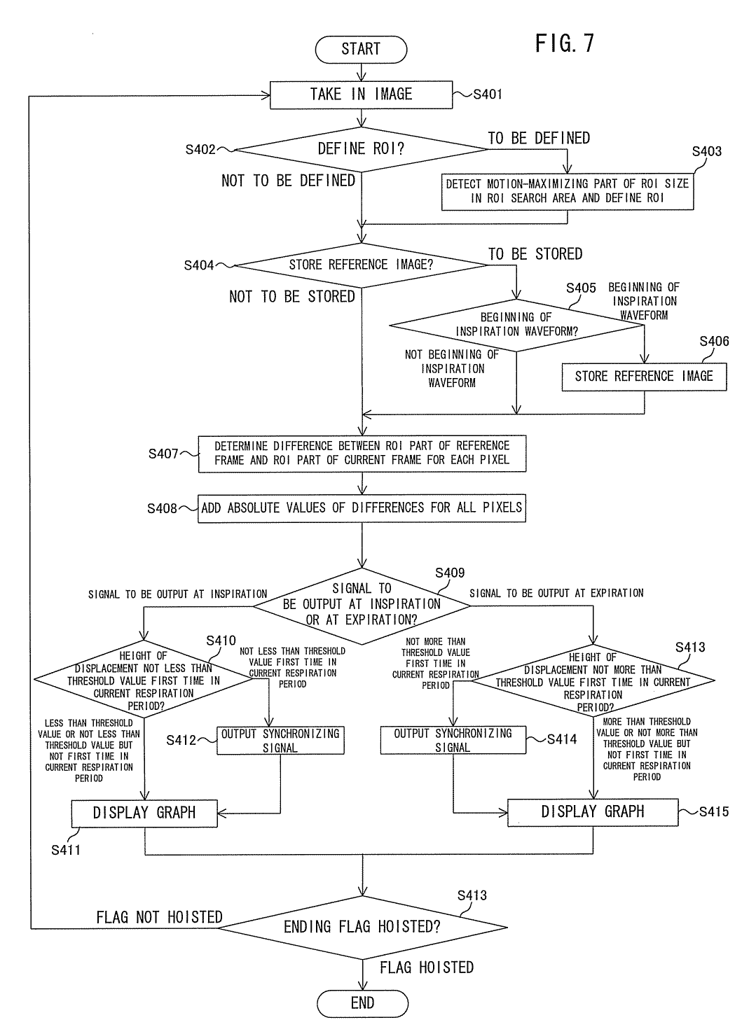

FIG. 7 is a flowchart of the process of the respiration monitoring apparatus (respiration monitoring method) according to the second embodiment. The steps S401 through S403 and S409 through S415 in FIG. 7 are same as the steps S101 through S103 and S109 through S116 in FIG. 4 and hence will not be described here any further.

When a reference image is to be stored (S404, store) after an imaging target region ROI is defined by the imaging region defining section 101 (S403), it is determined if the timing of the image pickup operation is at the beginning of the inspiration waveform or not (S405). If the timing is at the beginning of the inspiration waveform (the timing (reference clock time) when imaging target region gets to a predetermined limit position in the respiration), the picked up image is stored typically in the MEMORY 109 as reference image (S406). If, on the other hand, the timing is not at the beginning of the inspiration waveform or no reference image is to be stored (S404, not store), the process proceeds to the next processing step (S407).

Subsequently, the displacement computing section 103 computationally determines the difference of the luminance value of each of the pixels on the image acquired by the image acquiring section 102 at the arbitrary clock time that is an arbitrarily selected timing and the luminance value of the corresponding pixel on the image acquired by the image acquiring section 102 at the reference clock time (S407), and then determines the displacement of the position of the imaging target region from the reference clock time to the arbitrary clock time by adding the absolute values of the differences of all the pixels obtained as a result of the above process (displacement computing step) (S408).

Then, the position determining section 105 determines the displacement of the position of the imaging target region from the reference clock time to the arbitrary clock time as computationally determined by the displacement computing section 103 as the position of the imaging target region at the arbitrary clock time (position determining step).

Now, the process of the displacement computing section 103 will be described below in detail. When the coordinate value of a pixel in a direction substantially parallel to the height direction of the subject M is y, the coordinate value of the pixel in a direction substantially perpendicular to the height direction of the subject M is x, the reference clock time is t₀, the arbitrary clock time is tₙ and the luminance value of the pixel of the coordinates (x, y) at clock time t is I(x, y, t) in the imaging target region on the image acquired by the image acquiring section 102, the displacement Q_{b} of the position of the imaging target region between the reference clock time and the arbitrary clock time responding to the respiration of the subject M is computationally determined by formula:

Q_{b}=S S|(I(x,y,tₙ)−I(x,y,t₀))| (6),

(where the first S of the S S is the sum of the luminance values of all the pixels either in the y direction or in the x direction in the imaging target region ROI and the second S of the S S is the sum of the luminance values of all the pixels either in the x direction or in the y direction, whichever appropriate, in the imaging target region). The displacement computing section 103 of this embodiment computationally determines the displacement of the position of the imaging target region from the reference clock time to the arbitrary clock time according to the formula (6) above.

Thus, as described above, this embodiment computationally determines the difference of the luminance of the image acquired at the arbitrary clock time and that of the image acquired at the reference clock time so that, if compared with the technique of comparing the frame of an arbitrary clock time and the immediately preceding frame and computationally determining the difference, there is no risk of accumulating computation errors and hence this embodiment can accurately grasp the respiration of the subject.

### Third Embodiment

Now, the third embodiment of the present invention will be described below.

Since this embodiment is a modification to the above-described first embodiment, the component same as those of the above-described first embodiment are denoted by the same reference symbols and will not be described here any further. This embodiment differs from the above-described first embodiment in terms of the method of computing the displacement that the displacement computing section employs.

The imaging region defining section 101 of this embodiment is so adapted as to define an imaging target region ROI as a region centered at a pixel region that maximizes the temporal change of luminance of the pixels on the image obtained by shooting the subject M.

The displacement computing section 103 of this embodiment is adapted to extract the second region having pixels showing a luminance distribution substantially same as the first region having a plurality of arbitrarily selected pixels in the imaging target region ROI on the image acquired by the image acquiring section 102 at the first clock time that is an arbitrarily selected timing from the imaging target region ROI on the image acquired at the second clock time that is the timing of a predetermined number of counts as counted from the first clock time and computationally determine the distance of movement from the position of the first region to the position of the second region in the imaging target region ROI as the displacement of the position of the imaging target region from the first clock time to the second clock time.

The position determining section 105 of this embodiment is adapted to determine the position obtained by adding the displacement of the position of the imaging target region between the first clock time and the second clock time as computationally determined by the displacement computing section 103 to the position of the imaging target region at the first clock time as the position of the imaging target region at the second clock time.

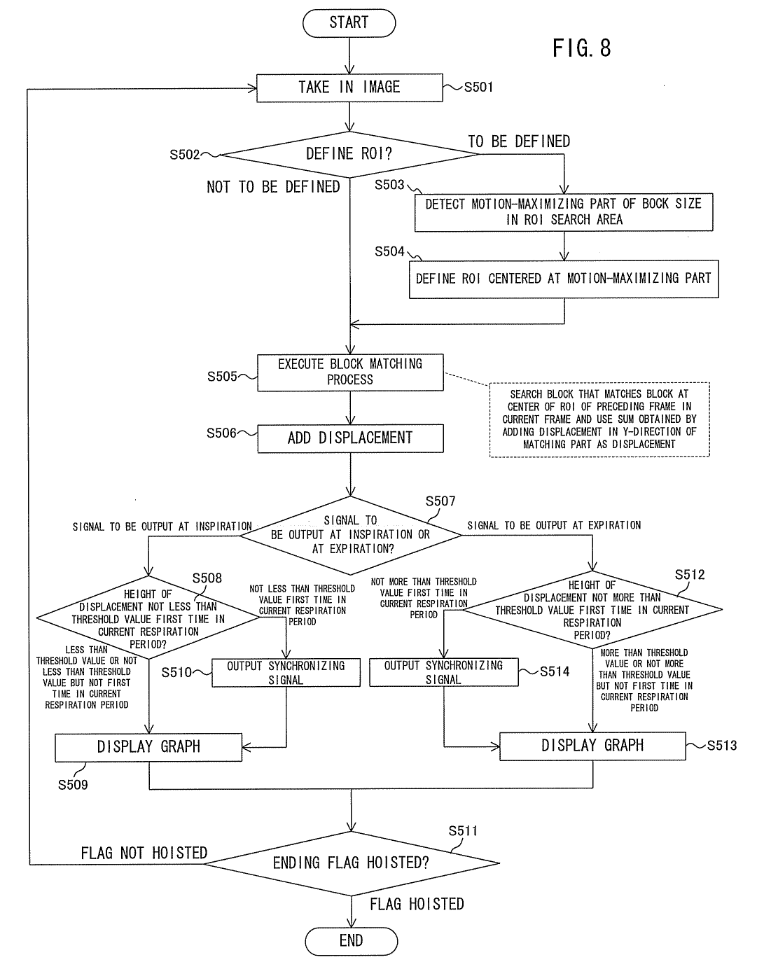

FIG. 8 is a flowchart of the process of the respiration monitoring apparatus (respiration monitoring method) according to the third embodiment. The steps S507 through S514 in FIG. 8 are same as the steps S109 through S116 in FIG. 4 and hence will not be described here any further.

Firstly, the image pickup section 2 picks up an image of a physical part (thorax or abdomen) that reciprocates in response to the respiration of the subject M and the surroundings thereof (S501).

Then, when an imaging target region ROI is to be defined (S502, to be defined), the region of a pixel region (block region) that maximizes the change with time of the luminance of the pixels on the image picked up by the image pickup section 2 by shooting the subject M is detected (S503) and a region centered at the pixel region is defined as imaging target region ROI (imaging region defining step) (S504). The image acquiring section 102 acquires an image picked up so as to show an inclination of a predetermined angle relative to the reciprocating direction of the defined imaging target region at each predetermined timing (image acquiring step).

Subsequently, the displacement computing section 103 extracts the second region having pixels showing a luminance distribution substantially same as the first region having a plurality of arbitrarily selected pixels in the imaging target region on the image acquired in the image acquiring step at the first clock time that is an arbitrarily selected timing from the imaging target region on the image (current frame) acquired at the second clock time that is the timing of a predetermined number of counts as counted from the first clock time and computationally determines the distance of movement from the position of the first region to the position of the second region in the imaging target region as the displacement of the position of the imaging target region from the first clock time to the second clock time (block matching process) (displacement computing step) (S505).

The position determining section 105 determines the position obtained by adding the displacement of the position of the imaging target region from the first clock time to the second clock time as computationally determined by the displacement computing section 103 to the position of the imaging target region at the first clock time as the position of the imaging target region at the second clock time (position determining step) (S506).

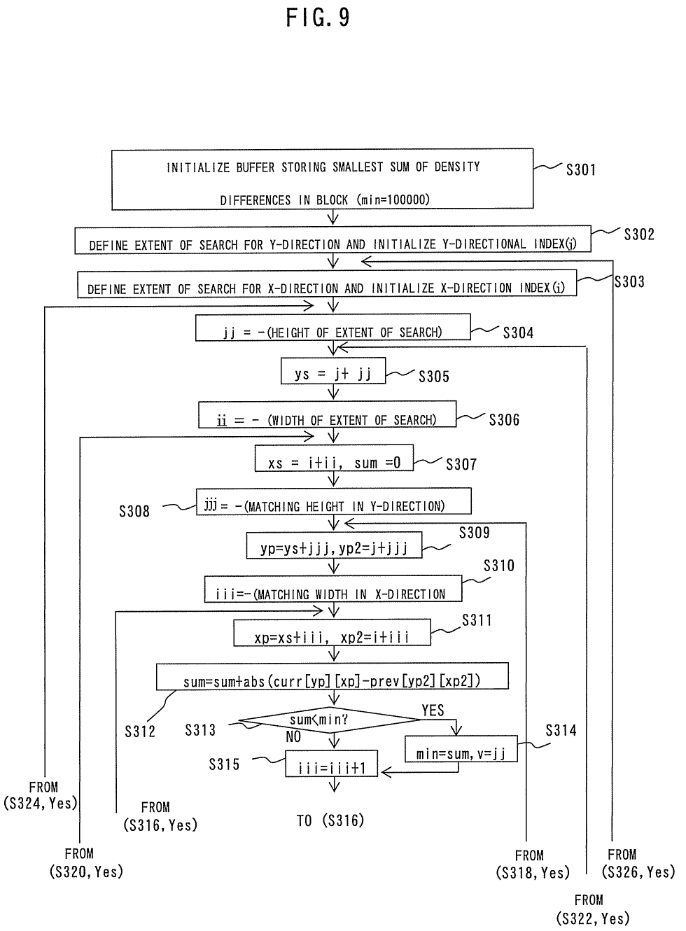

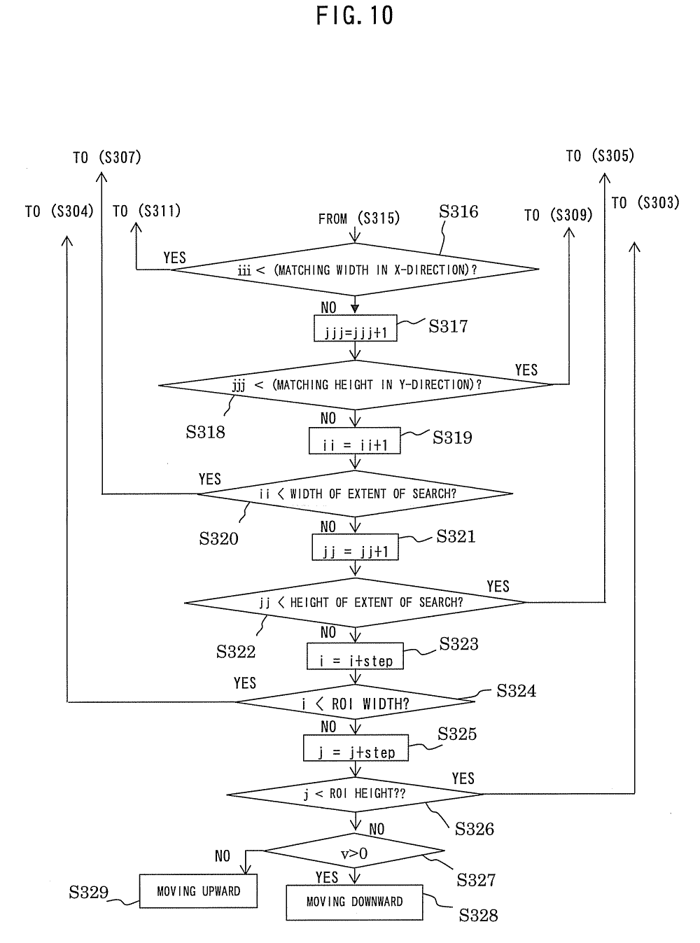

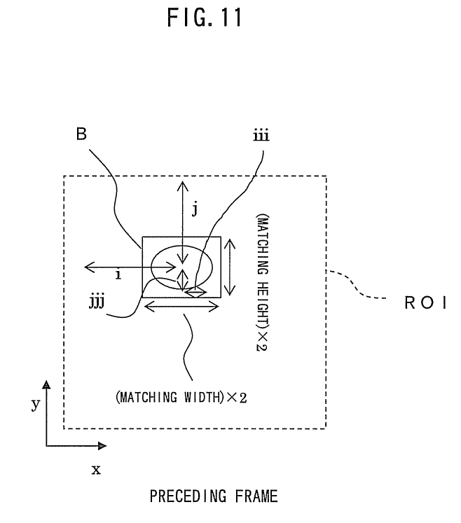

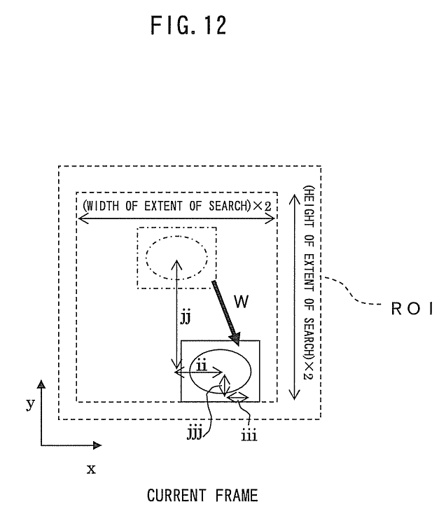

Now, the process of the displacement computing section 103 will be described below in detail. FIGS. 9 and 10 are flowcharts of the process of determining the moving directions of pixels on an image to be executed by the expiration/inspiration determining section 104 of this embodiment. Note that a single flowchart is divided into two flowcharts here for the sake of convenience. FIGS. 11 and 12 schematically illustrate the movement of a predetermined block on an image and a method of matching the block. Note that block B in the preceding frame is moved in the direction indicated by arrow W at the timing of the current frame.

As the technique of discriminating expiration and inspiration of this embodiment, in the imaging target region ROI in the image (current frame) acquired at a predetermined timing and the imaging target region ROI located at the same position in the image (preceding frame) acquired at the timing immediately preceding a predetermined timing, a plurality of rectangular blocks B (the first region) obtained by equally dividing the imaging target region ROI of the preceding frame in the X-direction and the Y-direction are defined, and the difference of density value between each of the pixels in each of the rectangular blocks of the preceding frame and corresponding one of the pixels in the same area near the position of the block in the current frame is determined and added on a block by block basis.

The pixel distribution of the block B in the preceding frame and that of the same area near the block in the current frame can be compared for accurate matching by moving the block B in the preceding frame in and near the area of the current frame where the block B was located by a unit smaller than the size of the block B at a time even if the block B has moved minutely. Of course, it is possible to move the block B by a unit equal to one of the plurality of rectangular blocks produced by equally dividing the imaging target region for matching.

The outcome of the addition process executed for each of the blocks in the preceding frame as described above is stored in the MEMORY 109. In this way, the above-described addition process is executed for all the blocks.

Then, when the block in the preceding frame and the block (second region) in the current frame that show the smallest sum of the differences of density value is detected, they show respective pixel patterns (patterns formed by pixels) that resemble each other most. Then, it may be safe to presume that the block in the preceding frame has moved to the position of the corresponding block in the current frame.

In this way, the object to be shot (the part to be examined) in the imaging target region ROI is presumed to have moved by the displacement of the position of the block. Then, the distance and the direction of movement of the block is expressed by a vector extending from a point in the block in the preceding frame to the corresponding point in the block in the current frame and if the vector is directed upward or downward is determined by seeing the sign of the Y-directional component of the vector.

Firstly, the buffer for storing the smallest sum of the differences of density value in the block defined in the imaging target region ROI is initialized (S301). For the algorithm employed in this embodiment, a value as large as possible is preferably taken for the numerical value that substitutes [min].

Then, the extent of search is defined for the Y-direction and the Y-directional index j is initialized (S302).

Thereafter, the extent of search is defined for the X-direction and the X-directional index i is initialized (S303).

Subsequently, the index for the height of the extent of search (index for defining the extent of search in the Y-direction) is initialized (S304).

The outcome of computation is stored in the buffer for the purpose of improving the efficiency of the process (S305).

Then, the index for the height of the extent of search (index for defining the extent of search in the X-direction) is initialized (S306).

The outcome of computation is stored in the buffer for the purpose of improving the efficiency of the process and the buffer for storing the sum of the differences of density value in the block is initialized (S307).

Then, the index for the matching height (the value that is equal to ½ of the size of the predetermined block B in the Y-direction and turned negative) is initialized (S308).

The outcome of computation is stored in the buffer for the purpose of improving the efficiency of the process (S309).

The index for the matching width in the X-direction (that value that is equal to ½ of the size of the predetermined block B in the X-direction and turned negative) is initialized (S310).

The outcome of computation is stored in the buffer for the purpose of improving the efficiency of the process (S311).

The sum of the absolute values of the differences of density value (differences of luminance values of pixels) in the predetermined block B is determined by addition (S312).

The smallest sum of the differences of density value in the predetermined block B and the sum of the differences of density value in each of the blocks are compared (S313).

The sum of the differences of density value in each of the blocks is stored in the buffer for storing the smallest sum of the differences of density value in the predetermined block B (S314).

Then, the index for the matching width is incremented (S315).

If the index for the matching width is smaller than the matching width in the X-direction or not is determined (S316).

Then, index for the matching height is incremented (S317).

Subsequently, if the index for the matching height is smaller than the matching height in the Y-direction or not is determined (S318).

The index for the width of the extent of search is incremented (S319).

Then, if the index for the width of the extent of search is smaller than the width of the extent of search or not is determined (S320).

The index for the height of the extent of search is incremented (S321).

Thereafter, if the index for the height of the extent of search is smaller than the height of the extent of search or not is determined (S322).

The extent of search is decided and the index for the X-direction is incremented (S323).

The extent of search is decided and if the index for the X-direction is smaller than the width of the ROI or not is determined (S324).

The extent of search is decided and the index for the Y-direction is incremented (S325).

The extent of search is decided and if the index for the Y-direction is smaller than the height of the ROI or not is determined (S326).

Then, the direction of change (temporal change) of the image in the imaging target region ROI is determined (S327). In this way, it is determined if the pixels in the imaging target region ROI are moving upward or downward (S329, S328).

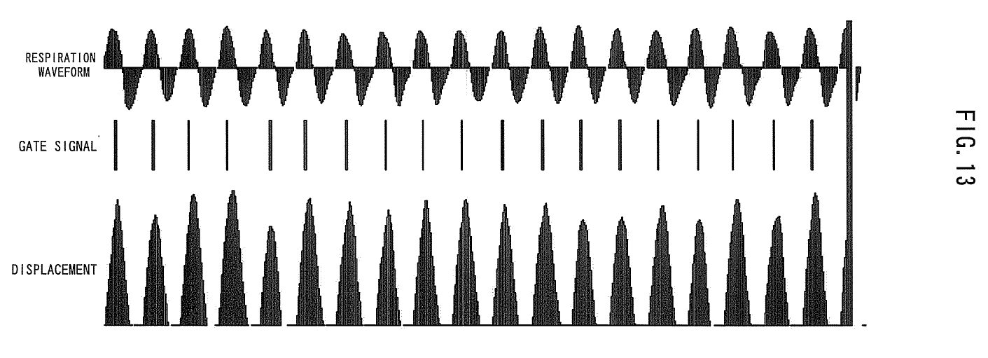

FIG. 13 is a schematic exemplar graph showing the position determined by the position determining section 105 as displayed on the display section 106.

As described above, this embodiment is adapted to execute a matching process, using block regions each having a plurality of pixels so that the displacement of the position of the imaging target region can be grasped accurately without being influenced by errors attributable to dispersion of luminance values of pixels and other factors. Additionally, since the imaging region defining section 101 defines an imaging target region centered at the pixel region showing the largest temporal change of luminance, a block matching process that is centered at the pixel region showing the largest temporal change of luminance can be selectively executed. Thus, the displacement of a physical part of a subject that reciprocates in response to the respiration of the subject can be highly accurately determined by computations.

### Fourth Embodiment

Now, the fourth embodiment of the present invention will be described below.

Since this embodiment is a modification to the above-described first embodiment, the component same as those of the above-described first embodiment are denoted by the same reference symbols and will not be described here any further. This embodiment differs from the above-described first embodiment in terms of the method of computing the displacement that the displacement computing section employs.

The displacement computing section 103 of this embodiment is so adapted as to extract the second region having pixels showing a luminance distribution substantially same as the first region having a plurality of pixels, whose number is arbitrarily selected, in an imaging target region ROI on the image acquired by the image acquiring section 102 at a reference clock time that is a timing when the imaging target region gets to a predetermined limit position in the respiration from the imaging target region ROI on the image acquired by the image acquiring section 102 at an arbitrary clock time that is an arbitrarily selected timing coming after the reference clock time and computationally determine the moving distance from position of the first region to the position of the second region as the displacement of the imaging target region from the reference clock time to the arbitrary clock time.

The position determining section 105 is adapted to determine the displacement of the position of the imaging target region between the reference clock time and the arbitrary clock time as computationally determined by the displacement computing section as the position of the imaging target region at the arbitrary clock time.

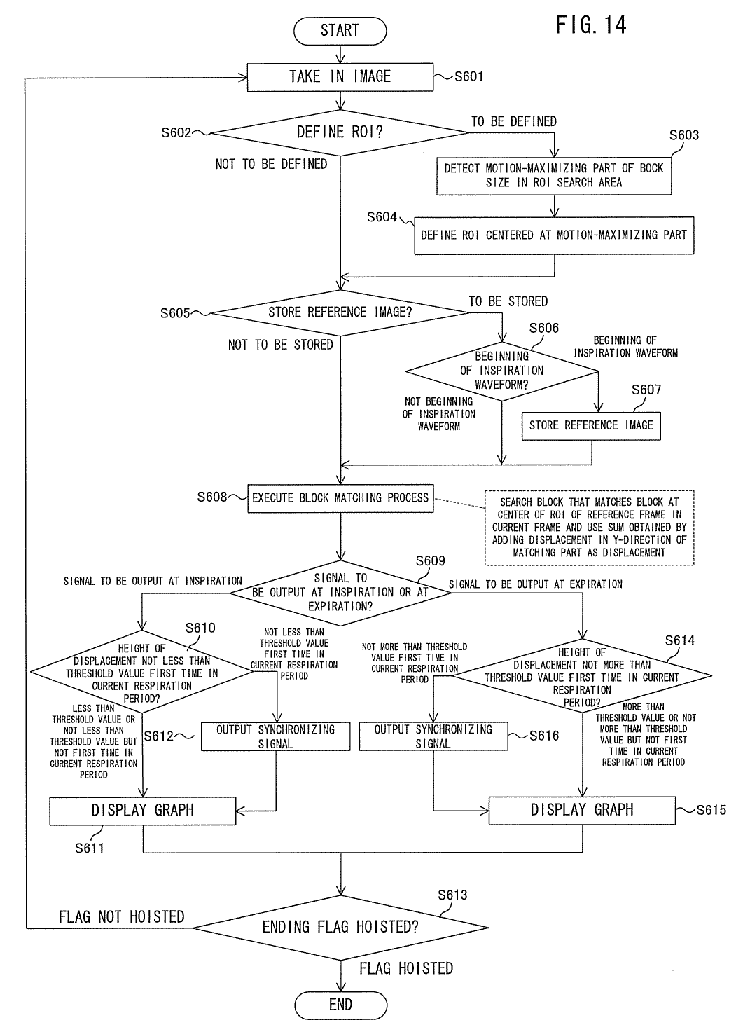

FIG. 14 is a flowchart of the process of the respiration monitoring apparatus (respiration monitoring method) according to the fourth embodiment. The steps S601 through S604 and S609 through S616 in FIG. 14 are same as the steps S501 through S504 and S507 through S514 in FIG. 8 and hence will not be described here any further.

When a reference image is to be stored (S605, store) after an imaging target region ROI is defined by the imaging region defining section 101 (S604), it is determined if the timing of the image pickup operation is at the beginning of the inspiration waveform or not (S606). If the timing is at the beginning of the inspiration waveform (the timing (reference clock time) when imaging target region gets to a predetermined limit position in the respiration), the picked up image is stored typically in the MEMORY 109 as reference image (S607). If, on the other hand, the timing is not at the beginning of the inspiration waveform or no reference image is to be stored (S605, not store), the process proceeds to the next processing step (S608).

Subsequently, the displacement computing section 103 extracts the second region having pixels showing a luminance distribution substantially same as the first region having a plurality of pixels, whose number is arbitrarily selected, in an imaging target region ROI on the image acquired by the image acquiring section 102 at a reference clock time that is a timing when the imaging target region gets to a predetermined limit position in the respiration from the imaging target region ROI on the image acquired by the image acquiring section 102 at an arbitrary clock time that is an arbitrarily selected timing coming after the reference clock time and computationally determines the moving distance from position of the first region to the position of the second region in the imaging target region ROI as the displacement of the imaging target region from the reference clock time to the arbitrary clock time (displacement computing step). The position determining section 105 determines the displacement of the position of the imaging target region between the reference clock time and the arbitrary clock time as computationally determined by the displacement computing section 103 as the position of the imaging target region at the arbitrary clock time (position determining step) (S608).

As described above, this embodiment is adapted to extract a region showing a luminance pattern same as a block region on the image acquired at an arbitrary clock time from the image acquired at a reference clock time. Thus, if compared with the above-described method of executing a block matching process of comparing a frame at an arbitrary clock time and the immediately preceding frame, this embodiment can accurately grasp the respiration of the subject without any risk of accumulating computation errors.

The steps of the process of the respiration monitoring method described above by referring to the embodiments are realized by causing the CPU 108 to execute the respiration monitoring program stored in the MEMORY 109.

While the functions for embodying the present invention are stored in advance in the inside of the apparatus in each of the above-described embodiments, the present invention is by no means limited thereto and similar functions may be downloaded to the apparatus from a network or installed into the apparatus from a recording medium storing the functions. Any recording medium that can store programs and is readable relative to the apparatus may be used for the purpose of the present invention. The functions that can be installed or downloaded in advance may be so adapted as to be realized as a result of cooperation with the OS (operating system) in the inside of the apparatus.

In the imaging region defining step of the respiration monitoring method described above by referring to each of the embodiments, a technique of defining an imaging target region that is a region having a predetermined number of pixels (corresponding to the ROI size) and maximizing the temporal change of luminance of the pixels on the image or a technique of detecting a pixel region (block region) that maximizes the temporal change of luminance of the pixels on the image and defining an imaging target region centered at the pixel region is employed. However, the present invention is by no means limited thereto and a technique as described below may alternatively be used for defining an imaging target region. Firstly, the image region obtained by shooting a subject is divided into small blocks, each having a predetermined number of pixels (e.g., 8×8 pixels), by means of a mesh and then the absolute values of the differences of luminance between the pixels on the image acquired at the first clock time that is an arbitrarily selected timing and the pixels on the image acquired at the second clock time that is the timing of a predetermined number of counts as counted from the first clock time are averaged for each of the small blocks so as to use the average value as the value (characteristic value) of the block. Thereafter, the value (characteristic value) of each of the small blocks of the entire image is multiplied by a matrix of three rows and three columns of

$$\begin{matrix}1 & 1 & 1 \\ 0 & 0 & 0 \\ - 1 & - 1 & - 1\end{matrix}$$

to execute a transversal emphasis process.

Subsequently, the region having a predetermined number of pixels and showing a long profile in a direction substantially orthogonal relative to the moving direction of the pixels due to respiration on the picked up image that maximizes the sum of the values obtained by way of the transversal emphasis process is searched for and the region detected as a result of the search is defined as the center of an imaging target region ROI. The region having a predetermined number of pixels and showing a long profile in a direction substantially orthogonal relative to the moving direction of the pixels due to respiration on the picked up image refers to a region having a size same as the ROI or the above-described block region in the direction substantially orthogonal relative to the moving direction of the pixels due to respiration and a height of a predetermined number of pixels (e.g. equal to two pixels) in the moving direction of the pixels due to respiration. A region showing a long profile in a direction substantially orthogonal relative to the moving direction of the pixels due to respiration is selected as reference because a region extending in a direction different from the moving direction of the pixels due to respiration and showing a strong contrast is effective when detecting the move of the pixels due to respiration.

As a result of defining an imaging target region that is centered at a region showing a large temporal change of luminance of the pixels on the image and a strong contrast (with light areas and dark areas that are clearly discernible), a region where the reciprocal motion of the thorax or the abdomen of the subject is likely to appear as a change of luminance can be selected as imaging target region. Thus, the reciprocal motion of the body surface that arises in response to the respiration of the subject can be monitored highly accurately. With the above-described technique of defining an imaging target region, a region that allows a pattern matching process to be executed with ease can be defined as ROI. Therefore, the above-described technique is particularly effective for a block matching process.

While a process of picking up an image by means of a CT scan apparatus is described above as an example of predetermined medical process that the medical process executing section causes to be executed, the present invention is by no means limited thereto and the predetermined medical process may alternatively be an image pickup process of an MRI (magnetic resonance imaging) apparatus that is another example of tomographic apparatus or a surgical process.

The displacement computing section computationally determines the displacement of the position of an imaging target region as a ratio (a scale for expressing the ratio of the displacement relative to the displacement waveform) relative to the displacement produced by a cycle of respiration of the subject (the displacement waveform of the thorax produced by respiration) so that the timing of executing a medical process can be specified by way of, for example, a position raised by 20% of the width of the reciprocation of the thorax (or the abdomen) from the position where the subject completely breathes out or a position lowered by 10% of the width of the reciprocation of the thorax (or the abdomen) from the position where the subject completes breathes in. In other words, “the position” determined by the position determining section of each of the above-described embodiments does not refer to the numerical value of the distance as expressed in centimeters, for example, by which the thorax or the abdomen rises.

However, it may be needless to say that the respiration monitoring apparatus of each of the above-described embodiments may be so adapted as to computationally determine the numerical value of the displacement of the thorax or the abdomen of the subject produced in response to the reciprocation as expressed in centimeters, for example, on the basis of the displacement of the pixels on the image or the position of arrangement or the angle of arrangement of the image pickup section.

While the image pickup section 2 is arranged so as to shoot the imaging target region ROI obliquely from above from the side of the feet of the subject M who is lying on the back in each of the above-described first and second embodiments, the present invention is by no means limited thereto. For example, the image pickup section 2 may alternatively be so arranged as to shoot the imaging target region ROI obliquely from above from the side of the head of the subject M who is lying on the back. With such an arrangement, the movement of the pixels on the image toward the side of the feet of the subject M (in the first direction) is determined as indication of inspiration, whereas the movement of the pixels on the image toward the side of the head of the subject M (in the second direction) is determined as indication of expiration. Of course, the image pickup section 2 may still alternatively be so arranged as to shoot the imaging target region ROI obliquely from above from a lateral side of the subject. In other words, what is necessary is to shoot the thorax or the abdomen of the subject M obliquely.

### INDUSTRIAL APPLICABILITY

As described above in detail, the present invention provides a technique of grasping the ratio of displacement of the position of a part of the body surface when the subject expires or inspires in a non-contact manner.

1. A respiration monitoring apparatus comprising:
an image acquiring section that acquires an image of an imaging target region including a physical part of a subject that reciprocates in response to the respiration of the subject as picked up with inclination of a predetermined angle relative to the reciprocating direction at each predetermined timing;
a displacement computing section that computationally determines the displacement of the position of the imaging target region between the first clock time that is an arbitrarily selected timing and the second clock time that is the timing of a predetermined number of counts as counted from the first clock time on the basis of the difference between the luminance of each of the pixels on the image acquired at the first clock time and the luminance of the corresponding pixel on the image acquired at the second clock time; and
a position determining section that determines the position obtained by adding the displacement of the position of the imaging target region between the first clock time and the second clock time as computationally determined by the displacement computing section to the position of the imaging target region at the first clock time as the position of the imaging target region at the second clock time.

2. The apparatus according to claim 1, wherein
when the coordinate value of a pixel in a direction substantially parallel to the height direction of the subject is y, the coordinate value of the pixel in a direction substantially perpendicular to the height direction of the subject is x, the first clock time is t₁, the second clock time is t₂ and the luminance value of the pixel of the coordinates (x, y) at clock time t is I(x, y, t) in the imaging target region on the image acquired by the image acquiring section, the displacement Q of the position of the imaging target region between the first clock time and the second clock time responding to the respiration of the subject is computationally determined by formula Q=S S|(I(x, y, t₂)−I(x, y, t₁))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

3. The apparatus according to claim 1, wherein
the image acquiring section is adapted to determine the moving direction of the pixels of image from the temporal displacement of the pixels on the images as acquired at a plurality of predetermined consecutive timings, the apparatus further comprising an expiration/inspiration determining section that determines the movement of the pixels on the images as that of inspiration when they move in the first direction as directional component on a plane defined by the height direction of the subject and a direction substantially parallel to the transversal direction relative to the subject but determines the movement of the pixels as that of expiration when they move in the second direction substantially opposite to the first direction;
the position determining section being adapted to add the displacement of the position of the imaging target region as displacement in expiration or in inspiration according to the outcome of determination by the expiration/inspiration determining section.

4. The apparatus according to claim 3, wherein
when the coordinate value of a pixel in a direction substantially parallel to the height direction of the subject is y, the coordinate value of the pixel in a direction substantially perpendicular to the height direction of the subject is x, the clock time is t and the luminance value of the pixel of the coordinates (x, y) at clock time t is I(x, y, t) in the imaging target region on the image acquired by the image acquiring section, the speed dy/dt of all the pixels in the imaging target region in the direction substantially parallel to the height direction of the subject is given by dy/dt=−(−S S((∂I(x, y, t)/∂x)*(∂I(x, y, t)/∂y))*S S((∂I(x, y, t)/∂t)*(∂I(x, y, t)/∂y))+S S(∂I(x, y, t)/∂x)²*S S((∂I(x, y, t)/∂y)*(∂I(x, y, t)/∂t)))/S S(∂I(x, y, t)/∂y)²*S S(∂I(x, y, t)∂x)²−(S S((∂I(x, y, t)/∂x)*(∂I(x, y, t)/∂y))²), (where the first S of the S S is the sum of the luminance values of all the pixels either in the y direction or in the x direction in the imaging target region, the second S of the S S is the sum of the luminance values of all the pixels either in the x direction or in the y direction, whichever appropriate, in the imaging target region), and
the expiration/inspiration determining section is adapted to determine that the direction of the speed of dy/dt indicates inspiration when it is directed in the first direction on the image and that it indicates expiration when it is directed in the second direction on the image.

5. A respiration monitoring apparatus comprising:
an image acquiring section that acquires an image of an imaging target region including a physical part of a subject that reciprocates in response to the respiration of the subject as picked up with inclination of a predetermined angle relative to the reciprocating direction at each predetermined timing;
a displacement computing section that computationally determines the displacement of the position of the imaging target region between an arbitrary clock time that is an arbitrarily selected timing and a reference clock time that is the timing for the imaging target region to get to a predetermined limit position in the respiration prior to the arbitrary clock time on the basis of the difference between the luminance of each of the pixels on the image acquired at the arbitrary clock time and the luminance of the corresponding pixel on the image acquired at the reference clock time; and
a position determining section that determines the displacement of the position of the imaging target region between the reference clock time and the arbitrary clock time as computationally determined by the displacement computing section as the position of the imaging target region at the arbitrary clock time.

6. The apparatus according to claim 5, wherein
when the coordinate value of a pixel in a direction substantially parallel to the height direction of the subject is y, the coordinate value of the pixel in a direction substantially perpendicular to the height direction of the subject is x, the reference clock time is t₀, the arbitrary clock time is tₙ and the luminance value of the pixel of the coordinates (x, y) at clock time t is I(x, y, t) in the imaging target region on the image acquired by the image acquiring section, the displacement Q_{b} of the position of the imaging target region between the reference clock time and the arbitrary clock time responding to the respiration of the subject is computationally determined by formula Q_{b}=S S|(I(x, y, tₙ)−I(X, y, t₀))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

7. The apparatus according to claim 1, further comprising:
an imaging region defining section that defines the region having a predetermined number of pixels that maximizes the temporal change of luminance of the pixels on the image obtained by shooting the subject as the imaging target region.

8. A respiration monitoring apparatus comprising:
an image acquiring section that acquires an image of an imaging target region including a physical part of a subject that reciprocates in response to the respiration of the subject as picked up with inclination of a predetermined angle relative to the reciprocating direction at each predetermined timing;
a displacement computing section that extracts the second region having pixels showing a luminance distribution substantially same as the first region having a plurality of arbitrarily selected pixels in the imaging target region on the image acquired by the image acquiring section at the first clock time that is an arbitrarily selected timing from the imaging target region on the image acquired at the second clock time that is the timing of a predetermined number of counts as counted from the first clock time and computationally determines the distance of movement from the position of the first region to the position of the second region in the imaging target region as the displacement of the position of the imaging target region from the first clock time to the second clock time; and
a position determining section that determines the position obtained by adding the displacement of the position of the imaging target region between the first clock time and the second clock time as computationally determined by the displacement computing section to the position of the imaging target region at the first clock time as the position of the imaging target region at the second clock time.

9. A respiration monitoring apparatus comprising:
an image acquiring section that acquires an image of an imaging target region including a physical part of a subject that reciprocates in response to the respiration of the subject as picked up with inclination of a predetermined angle relative to the reciprocating direction at each predetermined timing;
a displacement computing section that extracts from the imaging target region on the image acquired by the image acquiring section at an arbitrary clock time that is an arbitrarily selected timing the second region having pixels showing a luminance distribution substantially same as the first region having a plurality of arbitrarily selected pixels in the imaging target region on the image acquired by the image acquiring section at a reference clock time that is the timing for the imaging target region to get to a predetermined limit position in the respiration prior to the arbitrary clock time and computationally determines the distance of movement from the position of the first region to the position of the second region in the imaging target region as the displacement of the position of the imaging target region from the reference clock time to the arbitrary clock time; and
a position determining section that determines the displacement of the position of the imaging target region between the reference clock time and the arbitrary clock time as computationally determined by the displacement computing section as the position of the imaging target region at the arbitrary clock time.

10. The apparatus according to claim 8, further comprising:
an imaging region defining section that defines the imaging target region as a region centered at a pixel region that maximizes the temporal change of luminance of the pixels on the image obtained by shooting the subject.

11. A respiration monitoring system comprising:
the respiration monitoring apparatus according to claim 1; and
an image pickup section that picks up an image of an imaging target region from a position located obliquely above relative to the imaging target region at the side of the feet of the subject lying on the back.

12. A medical processing system comprising:
the respiration monitoring apparatus according to claim 1; and
a medical process executing section that causes a predetermined medical process to be executed when the position of the imaging target region as determined by the position determining section is located at a predetermined position.

13. The system according to claim 12, wherein
the predetermined medical process is an imaging process to be executed by means of MRI scan.

14. The system according to claim 12, wherein
the predetermined medical process is an imaging process to be executed by means of CT scan.

15. A respiration monitoring method comprising:
an image acquiring step that acquires an image of an imaging target region including a physical part of a subject that reciprocates in response to the respiration of the subject as picked up with inclination of a predetermined angle relative to the reciprocating direction at each predetermined timing;
a displacement computing step that computationally determines the displacement of the position of the imaging target region between the first clock time that is an arbitrarily selected timing and the second clock time that is the timing of a predetermined number of counts as counted from the first clock time on the basis of the difference between the luminance of each of the pixels on the image acquired at the first clock time and the luminance of the corresponding pixel on the image acquired at the second clock time; and
a position determining step that determines the position obtained by adding the displacement of the position of the imaging target region between the first clock time and the second clock time as computationally determined in the displacement computing step to the position of the imaging target region at the first clock time as the position of the imaging target region at the second clock time.

16. The method according to claim 15, wherein
when the coordinate value of a pixel in a direction substantially parallel to the height direction of the subject is y, the coordinate value of the pixel in a direction substantially perpendicular to the height direction of the subject is x, the first clock time is t₁, the second clock time is t₂ and the luminance value of the pixel of the coordinates (x, y) at clock time t is I(x, y, t) in the imaging target region on the image acquired in the image acquiring step, the displacement Q of the position of the imaging target region between the first clock time and the second clock time responding to the respiration of the subject is computationally determined by formula Q=S S|(I(x, y, t₂)−I(x, y, t₁))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

17. The method according to claim 15, wherein
the image acquiring step is adapted to determine the moving direction of the pixels of image from the temporal displacement of the pixel in the images as acquired at a plurality of predetermined consecutive timings, the method further comprising an expiration/inspiration determining step that determines the movement of the pixels on the images as that of inspiration when they move in the first direction as directional component on a plane defined by the height direction of the subject and a direction substantially parallel to the transversal direction relative to the subject but determines the movement of the pixels as that of expiration when they move in the second direction substantially opposite to the first direction, and
the position determining step being adapted to add the displacement of the position of the imaging target region as displacement in expiration or inspiration according to the outcome of determination in the expiration/inspiration determining step.

18. The method according to claim 17, wherein
when the coordinate value of a pixel in a direction substantially parallel to the height direction of the subject is y, the coordinate value of the pixel in a direction substantially perpendicular to the height direction of the subject is x, the clock time is t and the luminance value of the pixel of the coordinates (x, y) at clock time t is I(x, y, t) in the imaging target region on the image acquired in the image acquiring step, the speed dy/dt of all the pixels in the imaging target region in the direction substantially parallel to the height direction of the subject is given by dy/dt=−(−S S((∂I(x, y, t)∂x)*(∂I(x, y, t)/∂y))*S S((∂I(x, y, t)/∂t)*(∂I(x, y, t)/∂y))+S S(∂I(x, y, t)/∂x)²*S S((∂I(x, y, t)/∂y)*(∂I(x, y, t)/∂t)))/S S(∂I(x, y, t)/∂y)²*S S(∂I(x, y, t)∂x)²−(S S((∂I(x, y, t)∂x)*(∂I(x, y, t)/∂y))²), (where the first S of the S S is the sum of the luminance values of all the pixels either in the y direction or in the x direction in the imaging target region, the second S of the S S is the sum of the luminance values of all the pixels either in the x direction or in the y direction, whichever appropriate, in the imaging target region), and
the expiration/inspiration determining step is adapted to determine that the direction of the speed of dy/dt indicates inspiration when it is directed in the first direction on the image and that it indicates expiration when it is directed in the second direction on the image.

19. A respiration monitoring method comprising:
an image acquiring step that acquires an image of an imaging target region including a physical part of a subject that reciprocates in response to the respiration of the subject as picked up with inclination of a predetermined angle relative to the reciprocating direction at each predetermined timing;
a displacement computing step that computationally determines the displacement of the position of the imaging target region between an arbitrary clock time that is an arbitrarily selected timing and a reference clock time that is the timing for the imaging target region to get to a predetermined limit position in the respiration prior to the arbitrary clock time on the basis of the difference between the luminance of each of the pixels on the image acquired at the arbitrary clock time and the luminance of the corresponding pixel on the image acquired at the reference clock time; and
a position determining step that determines the displacement of the position of the imaging target region between the reference clock time and the arbitrary clock time as computationally determined in the displacement computing step as the position of the imaging target region at the arbitrary clock time.

20. The method according to claim 19, wherein
when the coordinate value of a pixel in a direction substantially parallel to the height direction of the subject is y, the coordinate value of the pixel in a direction substantially perpendicular to the height direction of the subject is x, the reference clock time is t₀, the arbitrary clock time is tₙ and the luminance value of the pixel of the coordinates (x, y) at clock time t is I(x, y, t) in the imaging target region on the image acquired in the image acquiring step, the displacement Q_{b} of the position of the imaging target region between the reference clock time and the arbitrary clock time responding to the respiration of the subject is computationally determined by formula Q_{b}=S S|(I(X, y, tₙ)−I(x, y, t₀))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

21. The method according to claim 15, further comprising:
an imaging region defining step that defines the region having a predetermined number of pixels that maximizes the temporal change of luminance of the pixels on the image obtained by shooting the subject as the imaging target region.

22. A respiration monitoring method comprising:
an image acquiring step that acquires an image of an imaging target region including a physical part of a subject that reciprocates in response to the respiration of the subject as picked up with inclination of a predetermined angle relative to the reciprocating direction at each predetermined timing;
a displacement computing step that extracts the second region having pixels showing a luminance distribution substantially same as the first region having a plurality of arbitrarily selected pixels in the imaging target region on the image acquired in the image acquiring step at the first clock time that is an arbitrarily selected timing from the imaging target region on the image acquired at the second clock time that is the timing of a predetermined number of counts as counted from the first clock time and computationally determines the distance of movement from the position of the first region to the position of the second region in the imaging target region as the displacement of the position of the imaging target region from the first clock time to the second clock time; and
a position determining step that determines the position obtained by adding the displacement of the position of the imaging target region between the first clock time and the second clock time as computationally determined in the displacement computing step to the position of the imaging target region at the first clock time as the position of the imaging target region at the second clock time.

23. A respiration monitoring method comprising:
an image acquiring step that acquires an image of an imaging target region including a physical part of a subject that reciprocates in response to the respiration of the subject as picked up with inclination of a predetermined angle relative to the reciprocating direction at each predetermined timing;
a displacement computing step that extracts from the imaging target region on the image acquired in the image acquiring step at an arbitrary clock time that is an arbitrarily selected timing the second region having pixels showing a luminance distribution substantially same as the first region having a plurality of arbitrarily selected pixels in the imaging target region on the image acquired at a reference clock time that is the timing for the imaging target region to get to a predetermined limit position in the respiration prior to the arbitrary clock time and computationally determines the distance of movement from the position of the first region to the position of the second region in the imaging target region as the displacement of the position of the imaging target region from the reference clock time to the arbitrary clock time; and
a position determining step that determines the displacement of the position of the imaging target region between the reference clock time and the arbitrary clock time as computationally determined in the displacement computing step as the position of the imaging target region at the arbitrary clock time.

24. The method according to claim 22, further comprising:
an imaging region defining step that defines the imaging target region as a region centered at a pixel region that maximizes the temporal change of luminance of the pixels on the image obtained by shooting the subject.

25. A respiration monitoring program for causing a computer to execute:
an image acquiring step that acquires an image of an imaging target region including a physical part of a subject that reciprocates in response to the respiration of the subject as picked up with inclination of a predetermined angle relative to the reciprocating direction at each predetermined timing;
a displacement computing step that computationally determines the displacement of the position of the imaging target region between the first clock time that is an arbitrarily selected timing and the second clock time that is the timing of a predetermined number of counts as counted from the first clock time on the basis of the difference between the luminance of each of the pixels on the image acquired at the first clock time and the luminance of the corresponding pixel on the image acquired at the second clock time; and
a position determining step that determines the position obtained by adding the displacement of the position of the imaging target region between the first clock time and the second clock time as computationally determined in the displacement computing step to the position of the imaging target region at the first clock time as the position of the imaging target region at the second clock time.

26. The program according to claim 25, wherein
when the coordinate value of a pixel in a direction substantially parallel to the height direction of the subject is y, the coordinate value of the pixel in a direction substantially perpendicular to the height direction of the subject is x, the first clock time is t₁, the second clock time is t₂ and the luminance value of the pixel of the coordinates (x, y) at clock time t is I(x, y, t) in the imaging target region on the image acquired in the image acquiring step, the displacement Q of the position of the imaging target region between the first clock time and the second clock time responding to the respiration of the subject is computationally determined by formula Q=S S|(I(x, y, t₂)−I(x, y, t₁))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

27. The program according to claim 25, wherein
the image acquiring step is adapted to determine the moving direction of the pixels of image from the temporal displacement of the pixel in the images as acquired at a plurality of predetermined consecutive timings, the method further comprising an expiration/inspiration determining step that determines the movement of the pixels on the images as that of inspiration when they move in the first direction as directional component on a plane defined by the height direction of the subject and a direction substantially parallel to the transversal direction relative to the subject but determines the movement of the pixels as that of expiration when they move in the second direction substantially opposite to the first direction, and
the position determining step being adapted to add the displacement of the position of the imaging target region as displacement in expiration or inspiration according to the outcome of determination in the expiration/inspiration determining step.

28. The program according to claim 27, wherein
when the coordinate value of a pixel in a direction substantially parallel to the height direction of the subject is y, the coordinate value of the pixel in a direction substantially perpendicular to the height direction of the subject is x, the clock time is t and the luminance value of the pixel of the coordinates (x, y) at clock time t is I(x, y, t) in the imaging target region on the image acquired in the image acquiring step, the speed dy/dt of all the pixels in the imaging target region in the direction substantially parallel to the height direction of the subject is given by dy/dt=−(−S S((∂I(x, y, t)/∂x)*(∂I(x, y, t)/∂y))*S S((∂I(x, y, t)/∂t)*(∂(x, y, t)/∂y))+S S(∂I(x, y, t)/∂x)²*S S((∂I(x, y, t)/∂y)*(∂I(x, y, t)/∂t)))/(S S(∂I(x, y, t)/∂y)²*S S(∂I(x, y, t)∂x)²−(S S((∂I(x, y, t)/∂x)*(∂I(x, y, t)/∂y))²), (where the first S of the S S is the sum of the luminance values of all the pixels either in the y direction or in the x direction in the imaging target region, the second S of the S S is the sum of the luminance values of all the pixels either in the x direction or in the y direction, whichever appropriate, in the imaging target region), and
the expiration/inspiration determining step is adapted to determine that the direction of the speed of dy/dt indicates inspiration when it is directed in the first direction on the image and that it indicates expiration when it is directed in the second direction on the image.

29. A respiration monitoring program for causing a computer to execute:
an image acquiring step that acquires an image of an imaging target region including a physical part of a subject that reciprocates in response to the respiration of the subject as picked up with inclination of a predetermined angle relative to the reciprocating direction at each predetermined timing;
a displacement computing step that computationally determines the displacement of the position of the imaging target region between an arbitrary clock time that is an arbitrarily selected timing and a reference clock time that is the timing for the imaging target region to get to a predetermined limit position in the respiration prior to the arbitrary clock time on the basis of the difference between the luminance of each of the pixels on the image acquired at the arbitrary clock time and the luminance of the corresponding pixel on the image acquired at the reference clock time; and
a position determining step that determines the displacement of the position of the imaging target region between the reference clock time and the arbitrary clock time as computationally determined in the displacement computing step as the position of the imaging target region at the arbitrary clock time.

30. The program according to claim 29, wherein
when the coordinate value of a pixel in a direction substantially parallel to the height direction of the subject is y, the coordinate value of the pixel in a direction substantially perpendicular to the height direction of the subject is x, the reference clock time is t₀, the arbitrary clock time is tₙ and the luminance value of the pixel of the coordinates (x, y) at clock time t is I(x, y, t) in the imaging target region on the image acquired in the image acquiring step, the displacement Q_{b} of the position of the imaging target region between the reference clock time and the arbitrary clock time responding to the respiration of the subject is computationally determined by formula Q_{b}=S S|(I(X, y, tₙ)−I(X, Y, t₀))|, (where the first S of the S S is the sum of the luminance values of all the pixels either in the y direction or in the x direction in the imaging target region and the second S of the S S is the sum of the luminance values of all the pixels either in the x direction or in the y direction, whichever appropriate, in the imaging target region).

31. The program according to claim 25, further comprising:
an imaging region defining step that defines the region having a predetermined number of pixels that maximizes the temporal change of luminance of the pixels on the image obtained by shooting the subject as the imaging target region.

32. A respiration monitoring program for causing a computer to execute:
an image acquiring step that acquires an image of an imaging target region including a physical part of a subject that reciprocates in response to the respiration of the subject as picked up with inclination of a predetermined angle relative to the reciprocating direction at each predetermined timing;
a displacement computing step that extracts the second region having pixels showing a luminance distribution substantially same as the first region having a plurality of arbitrarily selected pixels in the imaging target region on the image acquired in the image acquiring step at the first clock time that is an arbitrarily selected timing from the imaging target region on the image acquired at the second clock time that is the timing of a predetermined number of counts as counted from the first clock time and computationally determines the distance of movement from the position of the first region to the position of the second region in the imaging target region as the displacement of the position of the imaging target region from the first clock time to the second clock time; and
a position determining step that determines the position obtained by adding the displacement of the position of the imaging target region between the first clock time and the second clock time as computationally determined in the displacement computing step to the position of the imaging target region at the first clock time as the position of the imaging target region at the second clock time.

33. A respiration monitoring program for causing a computer to execute:
an image acquiring step that acquires an image of an imaging target region including a physical part of a subject that reciprocates in response to the respiration of the subject as picked up with inclination of a predetermined angle relative to the reciprocating direction at each predetermined timing;
a displacement computing step that extracts from the imaging target region on the image acquired in the image acquiring step at an arbitrary clock time that is an arbitrarily selected timing the second region having pixels showing a luminance distribution substantially same as the first region having a plurality of arbitrarily selected pixels in the imaging target region on the image acquired at a reference clock time that is the timing for the imaging target region to get to a predetermined limit position in the respiration prior to the arbitrary clock time and computationally determines the distance of movement from the position of the first region to the position of the second region in the imaging target region as the displacement of the position of the imaging target region from the reference clock time to the arbitrary clock time; and
a position determining step that determines the displacement of the position of the imaging target region between the reference clock time and the arbitrary clock time as computationally determined in the displacement computing step as the position of the imaging target region at the arbitrary clock time.

34. The program according to claim 32, further comprising:
an imaging region defining step that defines the imaging target region as a region centered at a pixel region that maximizes the temporal change of luminance of the pixels on the image obtained by shooting the subject.
